(19) United States
(12) Patent Application Publication
(10) Pub. No.: US 2026/0060834 A1
(43) Pub. Date: Mar. 5, 2026

(54) PATIENT POSITIONING DEVICE

(71) Applicant: Contract Medical Manufacturing LLC, Oxford, CT (US)

(72) Inventors: Richard Deslauriers, Woodbury, CT (US); Patrick Brandon, Killingworth, CT (US); Rodrigo DaSilva, Trumbull, CT (US)

(73) Assignee: Contract Medical Manufacturing LLC, Oxford, CT (US)

(21) Appl. No.: 19/349,182

(22) Filed: Oct. 3, 2025

Related U.S. Application Data
(63) Continuation-in-part of application No. 18/096,632, filed on Jan. 13, 2023, now Pat. No. 12,496,016.
(60) Provisional application No. 63/301,557, filed on Jan. 21, 2022.
(60) Provisional application No. 63/707,845, filed on Oct. 16, 2024.

(51) Int. Cl. A61F 5/37; A61G 13/12

(52) U.S. Cl. CPC A61F 5/3776; A61G 13/1225

(57) ABSTRACT

A patient positioning device attachable to a medical table comprising a base configured to overlie the table and underlie the patient, wherein the upper surface of the base faces the patient and the lower surface faces the table. One or more supports are removably attachable to the base to support a body region of the patient. The base can be composed of a foam material with a Velcro compatible material for removable attachment of the supports. Various shapes, sizes and function of the supports can be used.

This application claims priority from U.S. provisional application 63/707,845, filed Oct. 16, 2024, which is a continuation in part of application Ser. No. 18/096,632, filed Jan. 13, 2023, which claims priority from U.S. provisional application 63/301,557, filed Jan. 21, 2022. The entire contents of each of these applications are incorporated herein by reference.

### BACKGROUND

### Field of the Invention

This application relates to a patient positioning device, and, more particularly, to a patient positioning device attachable to an operating table to help secure the patient.

### Background

During various surgical procedures, patients are placed in different positions on the operating table such as a supine position, an inclined position, a reverse inclined position, a Trendelburg position (feet above the head), reverse Trendelburg position (tilted with head above the feet), etc. Several patient positioning devices of the prior art have been developed which are attachable to the operating table to help support the patient in the various positions. Their objective is to support the patient as well as restrain and/or immobilize the patient. Immobilization of the patient is especially important in robotic surgery because unwanted movement of the patient could require recalibration. The position of the patient could also aid the surgeon. However, each of the prior art patient positioning devices suffers one or more drawbacks such as limited versatility, compromised patient comfort, inadequate patient restraint, costly and/or difficult to manufacture, etc.

For example, one approach to hold the patient is the utilization of a foam pad. However, this pad has the risk of tearing. Further, the surface on the foam pad can give the patient a rash because of the high friction.

The need exists for an improved patient positioning device that provides increased comfort and improved patient restraint and enables better accommodation of patients of various sizes and in different positions on the operating table.

### SUMMARY

The patient positioning devices of the present invention overcome the problems and deficiencies of the prior art. The patient supporting devices of the present invention are placed on a medical table such as an operating table for supporting a patient in different positions. In some embodiments, the devices include a pad with bendable portions to form patient supports and limit movement of the patient on the operating table. In such embodiments, the bendable portions can also be utilized to attach the base to the medical table. In other embodiments, the patient supporting devices include a pad for selectively mounting a variety of patient supports that are preferably removably attachable to the pad. The positioning devices of the present invention have several advantageous features that enhance accommodation of patients of various sizes and in different positions on the operating table. The devices also have features providing increased versatility for attachment to the operating table as well as for securement of the patient to the table and support for various body regions of the patient. Each of these features are discussed in detail below.

In accordance with one aspect of the present disclosure, a patient positioning device attachable to a medical table such as an operating table or bed is provided. The patient positioning device comprises a base having an upper surface, a lower surface, a top region at a first portion, a bottom region at a second portion, a right-side region and a left side region. The base is configured to overlie the table and underlie the patient, wherein the upper surface of the base faces the patient and the lower surface faces the table. One or more supports are selectively removably attachable to the base to support a body region of the patient.

The supports can be of various sizes and shapes, and for various functions such as lumbar support, head or neck rest, shoulder support, etc. For example, the support(s) can comprise an arm rest removably attachable to one or both of the right or left side regions of the base, a head rest removably attachable to the top region of the base, a lumbar support attachable to a middle region of the base, etc.

In some embodiments, the base is made of a foam material and one or more portions of the base are bendable to form one or more bendable portions for attachment to a rail of the medical table. A support wire can be positioned within the bendable region to maintain a bent position of the bendable portion. The bendable portions in some embodiments can also be bent to provide arm or other body region support. In some embodiments, the bendable portions can be bent into a rolled position. The support wire in some embodiments is embedded in the foam material.

In some embodiments, the positioning device has a metal support attached to the base which is bendable to attach to the table. In some embodiments, the metal support is a single piece of metal; in other embodiments, the metal support comprises two or more metal members that are attached together. In either case, the metal support in such embodiments can extend along a length and a width of the base and can be arranged in an H-shape (longitudinal region and one or more transverse regions), a figure eight, X-shape or other shapes. In some embodiments, the metal support extends beyond right and left sides of a periphery of the base and are covered by a protective material. The protective material can be the same as the base and in some embodiments is a foam material.

In some embodiments, the base is asymmetric; in other embodiments, the base is symmetric so it can be positioned on the table in either direction and either side, i.e., the top and bottom regions are the same and the left and right side regions are the same.

In some embodiments, the positioning device includes a covering material covering at least a majority portion of the base to provide a Velcro friendly (compatible) material for removable attachment of the supports to the Velcro friendly material. In other embodiments, the positioning device itself has a Velcro friendly material.

In some embodiments, the base forms a first base and the device includes a second base attached to the first base, the first and second bases composed of a foam material and the metal support is sandwiched between the first and second bases. In some embodiments, a second covering material covers at least a majority portion of the second base to provide a Velcro friendly material for removable attachment of the supports to the Velcro friendly material if the device is used on a side where the second covering material faces the patient, the first base providing for the removable attachment of the supports if the device is used on a side where the first covering material faces the patient.

In accordance with another aspect of the present invention, a patient positioning device attachable to a medical table such as an operating table or bed, is provided. The patient positioning device comprises a base forming a first layer having a top region at a first portion, a bottom region at a second portion, a right side region and a left side region, and configured to overlie the table and underlie the patient to provide a support for the patient. A covering forming a second layer overlies a portion, and in preferred embodiments, at least a majority portion of the base and is configured to receive at least one patient support for attachment thereto. In some embodiments, the covering material provides a Velcro attachment surface for the at least one patient support.

In some embodiments, the positioning device includes a third layer, preferably of the same material as the first layer, and a second covering material forming a fourth layer covering the third layer, the first and third layers attachable to each other wherein the third and fourth layers are exposed and provide a surface for contacting the patient and for receiving the patient support(s).

In some embodiments, the base can comprise a foam pad having a plurality of slits formed therein to form zones of differing response to loads.

In some embodiments, the positioning device can include an absorbent material positioned over the base to provide a high-water absorption rate.

In accordance with another aspect of the present invention, a patient positioning device attachable to a medical table is provided comprising a base having a top region at a first portion, a bottom region at a second portion, a right side portion and a left portion. The base is configured to underlie the patient to provide a support for the patient. A metal support is attached to the base, the metal support including one or more metal elements that extend along a length and a width of the base, the metal supports bendable for attachment to a rail of the table.

In some embodiments, the base is composed of a foam which includes a plurality of slits to form zones of differing response to loads. In some embodiments, the metal support has a first section extending from the top region to the bottom region and a second section transverse to the first region extending from the left side region to the right side region, and in some embodiments extends beyond the left and right side regions.

In accordance with another aspect of the present invention, a patient positioning device attachable to a medical table is provided comprising a) a base having a top region at a first portion, a bottom region at a second portion, a right side portion and a left portion, the base configured to overlie the table and underlie the patient to provide a support for the patient; and b) a lumbar support removably attachable to the base, the lumbar support including a first channel and a second channel, each channel configured to receive and secure an arm of the patient. The channels are open and provide arm capture with direct visualization of the IV site.

In some embodiments, the lumbar support comprises a first support member and a second support member spaced from the first support member, wherein the first channel is in the first support member and the second channel is in the second support member.

In some embodiments, the lumbar support has a first end portion, a second end portion and an intermediate portion between the first and second end portions, the first and second end portions extending a further distance from the base than the intermediate portion. In some embodiments, the first channel is in the first end portion and the second channel is in the second end portion.

The positioning device can in some embodiments further include a) a head support removably attachable to the base, the head support including a cradle configured to receive a head of the patient and a cutout configured to receive a face of the patient when the patient is in a downwardly facing position; and/or b) first and second shoulder supports removably attachable to the base.

In some embodiments, the base comprises a foam material and a Velcro-friendly material, wherein the lumbar support has a Velcro strip on a bottom surface to removably attach to the Velcro-friendly material.

In some embodiments, the base comprises a foam including a colorant to reduce shifting color such as UV yellowing. Such shifting color can be caused by oxidation which can occur for example post sterilization. The tint of the colorant prevents or at least limits such discoloring to maintain aesthetics of the pad.

The positioning device can include first and second straps removably attachable to the base, each strap looping through rails of the medical table and attaching to itself to secure the base to the bed. The positioning device can further include first and second patient securing straps, each strap removably attachable to the base and extending over the patient and attaching to itself to secure the patient to the bed. In other embodiments, the positioning device can include one or more straps which wrap around metal rails of the table and attach to themselves to secure the device to the table or positioned on top to secure the patient to the table.

In some embodiments, the positioning device is packaged along with multiple other positioning devices in a vacuum sealed packaging.

In accordance with another aspect the present invention, a patient positioning device attachable to a medical table is provided, the patient positioning device comprising a) a base having an upper surface, a lower surface, a top region at a first portion, a bottom region at a second portion, a right side region and a left side region, the base configured to overlie the table and underlie the patient, wherein the upper surface faces the patient and the lower surface faces the table, the base composed of a foam material and a Velcro compatible material on an upper surface; and b) first and second straps attached to an upper surface of the Velcro compatible material, the straps stretchable and wrap around the medical table.

In some embodiments, the Velcro compatible material is integrated with the foam; in other embodiments the Velcro compatible material is adhesively secured to the foam.

In some embodiments, the straps have Velcro adhering material at each end for attachment to each other.

The positioning device can further include at least one patient strap attachable to the Velcro friendly surface and configured to be wrapped around the patient. The at least one patient strap can have Velcro adhering material at each end for attachment to each other. The at least one patient strap in some embodiments can be used in the other direction to wrap to the table rails.

In accordance with another aspect of the present invention, a patient positioning device attachable to a medical table is provided comprising a base having a top region at a first portion, a bottom region at a second portion, a right side region and a left side region, the base configured to overlie the table and underlie the patient to provide a support for the patient, the top region including a Velcro-friendly material, wherein the at least one support comprises a lumbar support having a first end portion, a second end portion and an intermediate portion between the first and second end portions, the first and second end portions extending a further distance from the base than the intermediate position to provide raised lateral edges to limit side to side rolling of the patient.

In some embodiments, the lumbar support comprises a first and second slots (channels) to receive right and left arms of a patient. In some embodiments, the lumbar support includes at least one area of Velcro for attachment to the Velcro-friendly material.

### BRIEF DESCRIPTION OF THE DRAWINGS

Preferred embodiment(s) of the present disclosure are described herein with reference to the drawings wherein:

FIG. 1A is a perspective view of a first embodiment of the patient positioning device of the present invention, the side arm rest sections shown bent upwardly and the arm straps shown in the looped position to secure the bent side sections;

FIG. 1B is a perspective view of an alternate embodiment of the patient positioning device of the present invention having a base with a single left and right top bendable portion (shown in the unbent position), and further showing the lower bendable portions bent downwardly;

FIG. 1C illustrates the head piece of FIG. 1B being removed for repositioning on the base of the device;

FIG. 1D is a perspective view illustrating the top (upper) outer sections of the base of FIG. 1A bent downwardly and the top (upper) inner sections of the base in a rolled position;

FIG. 2A is a perspective transparent view of the device of FIG. 1A, the device shown transparent to illustrate the metal strips and the friction pads on the underside (lower/bottom surface) of the base of the device, and the device shown with the side sections in the planar position;

FIG. 2B is a view similar to FIG. 2A showing the side arm sections bent and arm straps looped as in FIG. 1A;

FIG. 3 is a perspective view showing the underside (lower/bottom surface) of the device of FIG. 1A;

FIG. 4 is a top perspective view of an alternate embodiment of the device of the present invention showing the arm straps in the upward position and the side arm sections in the planar position;

FIG. 5 is a top perspective view of the device of FIG. 4 with the arm straps and head rest removed for clarity;

FIG. 6 is a perspective view of an alternate embodiment showing detachment of one of the side arm rest sections from the base of the device of FIG. 1A;

FIG. 7 is a perspective view showing the side arm sections attached to the base of the device of FIG. 1A;

FIG. 8 is a close-up view of the area of detail identified in FIG. 7 showing the tear apart connection of the side arm section and base;

FIG. 9 is a perspective view showing the left arm section being detached from the base of the device of FIG. 7;

FIG. 10 is a perspective view of examples of various shaped supports or modules which can be used for positioning regions of the body including body extremities;

FIG. 11 is a bottom view of an alternate embodiment of the device having a foam core (base) covered by a Velcro friendly layer (metal H-support not shown);

FIG. 12 is a transverse cross-sectional view of the device of FIG. 11 showing the base and covering layer;

FIG. 13 is a bottom view of the device of FIG. 11 showing one embodiment of a metal support attached thereto;

FIG. 14 is a bottom perspective view of the device of FIG. 13;

FIG. 15A is a perspective view showing the metal support sandwiched between two foam pads in accordance with a dual layer embodiment;

FIG. 15B is a perspective view similar to FIG. 15A showing the foam pad extending over the extending ends of the metal support and further showing a lumbar support attached to the base via attachment to the covering material;

FIG. 16 is a transverse cross-sectional view of the dual layer positioning device of FIG. 15A;

FIG. 17 is a top perspective view of an alternate embodiment of the patient positioning device (system) of the present invention showing various supports attached to the base;

FIG. 18 is a top view of the base of FIG. 17A without supports and showing the dimensions;

FIGS. 19A, 19B and 19C are top, side and bottom views, respectively, of the bedrail straps of the device of FIG. 17;

FIGS. 20A, 20B and 20C are top, side and bottom views, respectively, of the patient straps of the device of FIG. 17;

FIG. 21A is a perspective view of the shoulder bump of the device of FIG. 17;

FIGS. 21B and 21C are top and bottom views, respectively, of the shoulder bump of FIG. 21A;

FIG. 21D is a front view of the shoulder bump of FIG. 21A;

FIG. 22A is a perspective view of the head support of the device of FIG. 17;

FIG. 22B is a bottom view of the head support of FIG. 22A;

FIG. 22C is a front view of the head support of FIG. 22A;

FIG. 23A is a perspective view of the lumbar cradle of the device of FIG. 17;

FIG. 23B is a bottom view of the lumbar cradle of FIG. 23A;

FIG. 24 is a perspective view of an alternate embodiment of the lumber cradle having arm slots;

FIG. 25 is a perspective view similar to FIG. 17 except showing the lumbar cradle of FIG. 24 positioned on the patient positioning device;

FIG. 26 is a top perspective view of an alternate embodiment of the lumber cradle having arm slots;

FIG. 27 is top perspective view of a portion of the base of the device of FIG. 17 showing the lumbar cradle of FIG. 26 positioned on the base;

FIG. 28 is a top perspective view of an alternate embodiment of the lumbar cradle having arm slots;

FIG. 29 is a top perspective view of a portion of the base of FIG. 17 showing the lumbar cradle positioned on the base;

FIG. 30 is a bottom perspective view of the lumbar cradle of FIG. 25;

FIG. 31 is a top perspective view of an alternate embodiment of the patient positioning device of the present invention having an open head piece and shoulder/arm pieces;

FIG. 32 is a perspective view of a knee distraction device of the prior art with a close up view of the doghouse and bone distraction.

FIG. 33 is a perspective view of a knee distraction device of FIG. 32.

FIG. 34 is a perspective view of an embodiment of the doghouse of the present invention for knee distraction; and

FIG. 35 is a cross-sectional view of the doghouse of FIG. 34.

### DETAILED DESCRIPTION OF PREFERRED EMBODIMENTS

The present invention provides patient positioning devices that are attachable to a patient supporting structure such as an operating table or bed to support the patient on the table or bed. In some embodiments, the devices include pads with bendable portions to form patient supports and limit movement of the patient on the operating table. In other embodiments, the devices include pads to provide a mount for a variety of patient supports which are removably attachable to the pads. The devices have several advantageous features that enhance accommodation of patients of various sizes and in different positions on the table as well as provide improved patient securement and improved patient restraint/stabilization in a cost effective manner.

In general, several versions of the devices are disclosed. In one version, the device is in the form of a pad with bendable portions which provide patient support and table mounting structure. In another version, the device is in the form of a pad onto which patient supports of various shapes, sizes and functions can be selectively, and preferably removably, attached. Each of these versions have several alternative embodiments which are discussed in detail below.

The positioning devices of the bendable portion versions of the present invention (referred to herein for convenience as Version I) have several features to enhance patient comfort and/or securement and include one or more of the following: a) removable arm rests; b) bendable foam portions that are maintained in a bent condition; c) straps extending through the foam portions; d) foldable portions for shoulder comfort; and/or e) a slidable/repositionable head rest. Each of these features are discussed in detail below. Note that the positioning devices of the present invention can have all of the foregoing features a-e, or fewer than all such as one or two of these features, and still provide advantages over current patient positioning devices. Other features of the devices will become apparent from the discussion below.

The positioning devices of the mountable support versions of the present invention (referred to herein for convenience as Version II) have several features to enhance patient comfort and/or securement and include one or more of the following: a) modules removably attachable to the upper/top surface of the device where desired by the clinician to support and/or reposition the patient's body; b) a covering layer or a material attached to, or integrated with, the pad (base) providing an increased mounting surface area for the modules; and/or c) a bendable metal support and/or straps attached to the base to secure the patient and/or attach to the operating table to secure the device. Each of these features are discussed in detail below. Note that the positioning devices of the present invention can have all of the foregoing features a-c, or fewer than all such as one or two of these features, and still provide advantages over current patient positioning devices. Other features of the devices will become apparent from the discussion below.

Note that various features of one version can be used with the other version so the present invention is not limited to the features of the single version. For example, the bendable portion version (Version I) could include the metal support of Version II, the bendable Version I could include the removably attachable modules/supports of Version II, the bendable Version I could include the cover of Version II; the mountable support version (Version II) could include one or more of the bendable portions of Version I, etc.

The patient positioning devices of the present invention can advantageously be used to comfortably support and stabilize patients (provide stable positioning) on the operating table in various positions such as a supine position, an inclined position with respect to the horizontal, a reverse inclined (declined) position with respect the horizontal, a Trendelburg position (feet above the head), reverse Trendelburg position (tilted with head above the feet), etc. Other patient positions are also contemplated.

The positioning devices of the present invention can advantageously accommodate patients of varying BMI, e.g., thin, medium, and large.

The bendable sections of the positioning device of the bendable version described in detail below support and stabilize the patient and thus reduce or eliminate reliance on foam friction to stabilize the patient.

Note that the term “patient positioning device” includes the base/supporting foam material as well as can include the attachable modules/supports. Thus, even if the modules/supports are provided separately, e.g., packaged or sold separately, they can be considered part of the patient positioning device since they are mounted to the base and assist patient positioning. Thus, the “patient positioning device” can also be considered a “patient positioning system” and “patient positioning device” and “patient positioning system” as used herein can be considered interchangeable.

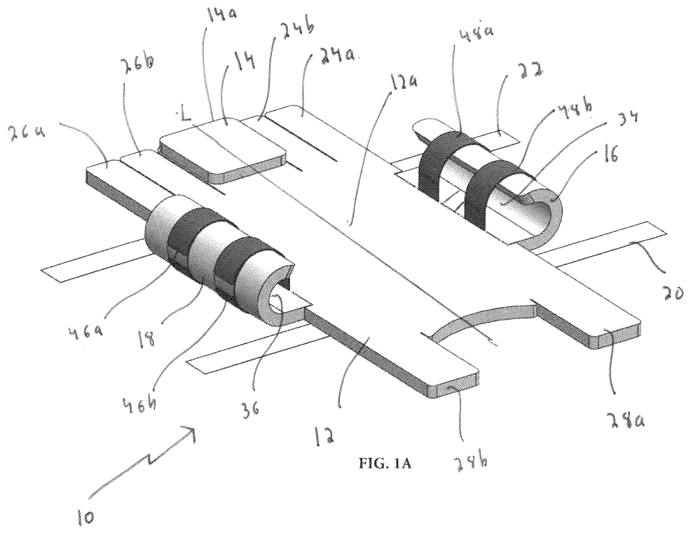

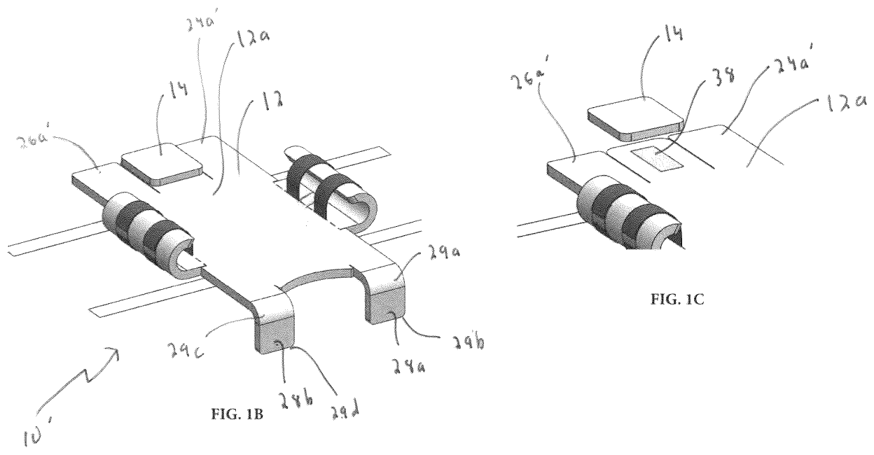

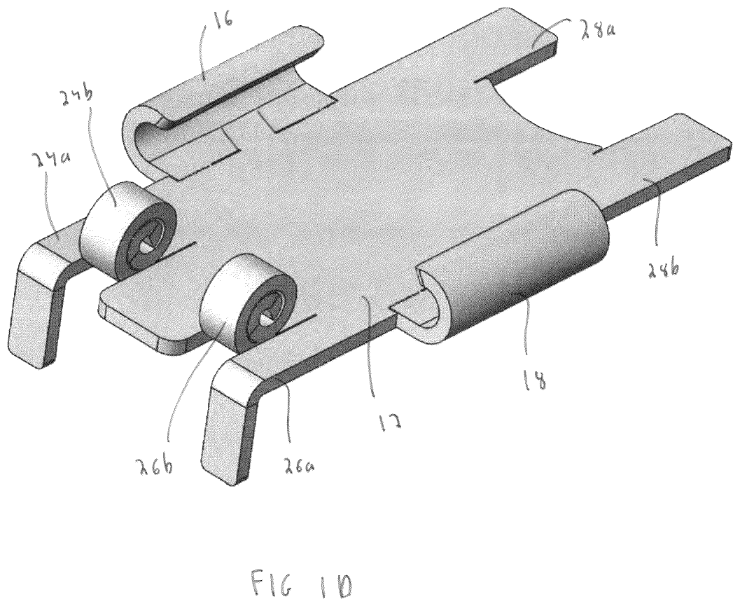

Referring now to the drawings and particular embodiments of the present invention wherein like reference numerals identify similar structural features of the devices disclosed herein, there are illustrated in FIGS. 1A-31 several embodiments of the patient positioning devices/systems of the present invention. The positioning devices are configured for attachment to the operating table. FIGS. 1A-9 illustrate the bendable portion version (Version I) and FIGS. 11-31 illustrate the module attachment (mountable support) version (Version II). It should be appreciated that features of one version can be used with the other version.

With initial reference to FIG. 1A, the positioning device/system is designated generally by reference numeral 10 and includes a main base 12 which forms the main portion of the device 10 and is preferably composed entirely of foam or the core composed of foam, although other materials are also contemplated. The foam can be a memory foam and can be an open pore or closed pore design. In other preferred embodiments, a non-memory foam is utilized. A floating head rest 14 is removably positioned on the base 12 and can be repositioned along the top region of the base 12. Head rest 14 is shown as substantially rectangular in shape with a substantially planar upper surface, however, other configurations are also contemplated. Left arm section 16 and right arm section 18 extend outwardly (transversely) from the left and right side portions, respectively, of the base 12. In some embodiments, as described in detail below, the arm sections 16, 18 are removably attached to the base 12. Note the head rest 14 and left and right arm sections 16, 18, like the base 12, are preferably composed of a foam material.

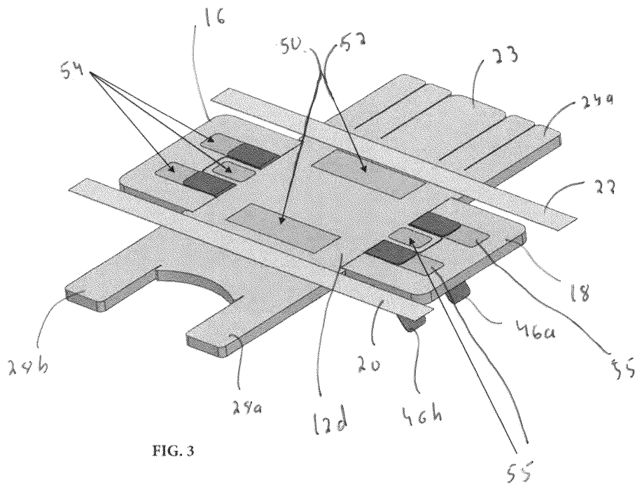

Note, as used herein, the term “top” and “bottom” and “left” and “right” refer to the orientation corresponding to the position of the patient resting/lying on the positioning device. With the patient lying facing upwardly, the patient's back rests against or faces toward on upper surface 12a of base 12 and the patient's head at the “top” portion (region) of the base 12. Stated another way, the upper surface 12a of the base faces the patient. The “lower” surface of the base 12, which rests against or faces the top surface of the operating table (not shown) or operating table pad, is shown in FIG. 3 and is designated by reference numeral 12d. Stated another way, the lower surface 12d of the base 12 faces the operating table. With the patient facing upwardly, the patient's right arm rests on, or is adjacent, “right” section 18 and the patient's left arm rests on, or is adjacent, “left” section 16. In preferred embodiments, the right and left sections are symmetrical, although asymmetrical configurations are also contemplated.

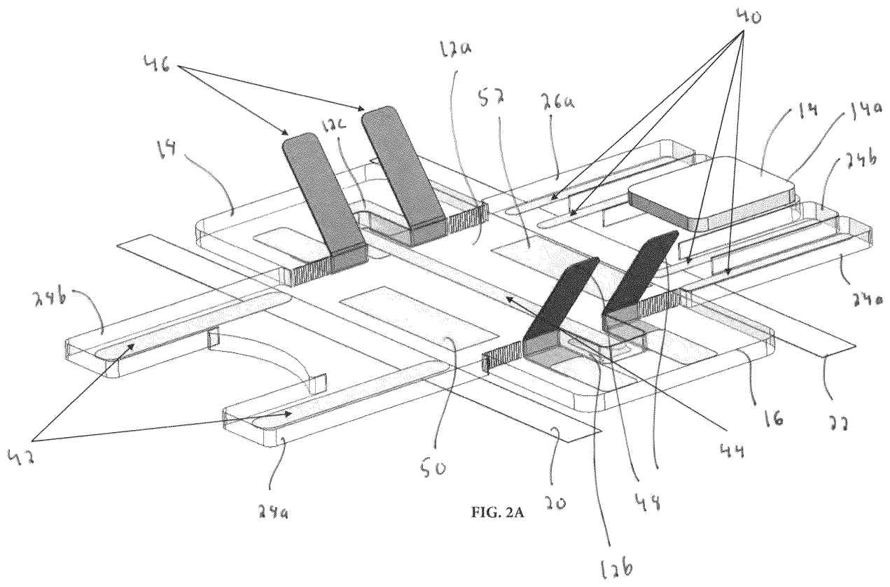

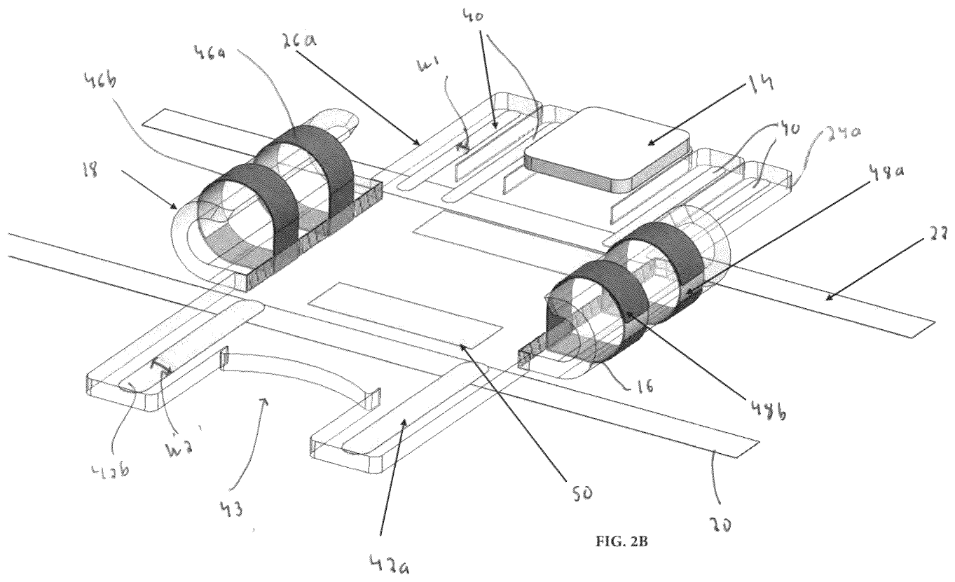

The bottom region (portion/section) of base 12 includes left and right extensions 28a, and 28b. These extensions (sections) are shown in the parallel position in FIGS. 1A, 2A and 2B, although non-parallel positions are also contemplated. In this parallel position, the extensions 28a, 28b are in a non-bent linear (planar) position. In the embodiment of these Figures, the extensions 28a, 28b are bendable downwardly toward the underlying operating table, out of the plane of the base 12, to help secure the bottom end of the base 12 to the operating table as it can engage a rail or the bed of the table. FIG. 1B, which illustrates an alternate embodiment of the positioning device as described below, shows the extensions 28a, 28b in the downwardly bent position, bent at regions 29a, 29c and terminating in ends 29b, 29d, respectively. The extensions 28a, 28b of FIG. 1A can be bent in a similar manner. The sections 28a, 28b in preferred embodiments are independently bendable although alternatively they can be co-joined to bend together. In some embodiments, the extensions 28a, 28b, can be bendable upwardly.

Each bendable extension 28a, 28b can include a bendable metal strip 42 embedded (captured) inside the foam, shown in the transparent view of FIG. 2A. The metal strip 42 provides support to maintain (and reinforce) the respective extension 28a, 28b in the bent position. In some embodiments, the metal strips 42 are 0.032 inches thick, although other dimensions (e.g., thicknesses) are also contemplated. In the absence of such metal strips/metal supports, the foam material would not stay in the bent position and would tend to return towards (and potentially into) the straight/non-bent position of FIG. 1A.

Note in alternate embodiments, sections 28a, 28b are not bendable and remain in the linear/parallel position. In other embodiments, the extensions (and bendable sections) are made of a material that can stay in the bent position without a metal support.

The top portion (region/section) of base 12 includes a pair of linear sections 24a, 24b on the left side of the head rest 14 and a pair of linear sections 26a, 26b on the right side of head rest 14. These sections 24a, 24b, 26a, 26b can be formed by elongated cuts in the foam which create these separate sections to enable independent bendability. In preferred embodiments, the sections 24a, 24b, 26a, 26b would extend slightly past the topmost edge (end) 14a of the head rest 14. In some embodiments, they can extend 6 inches past the topmost end 14a, although other lengths/distances are also contemplated, such as terminating at the topmost end 14a or terminating before the topmost end 14a. Note section 24b is inward of section 24a and section 26b is inward of section 26a, inward defined as closer to the central longitudinal axis L of the base 12.

The sections 24a, 24b, 26a, 26b are shown in the parallel position in FIGS. 1A, 2A and 2B. In this parallel position, these sections 24a, 24b, 26a, 26b are linear/non-bent extending in the same plane as the base 12. In some embodiments, each of the sections 24a, 24b, 26a, 26b are additionally or alternatively bendable downwardly to wrap around the operating table, e.g., a rail, to help secure device 10 to the operating table. In some embodiments, the sections 24a, 24b, 26a, 26b are additionally or alternatively bendable upwardly to provide a contact portion for the shoulders of the patient, and can be rolled as shown in FIG. 1D to provide a contact/abutment section for the patient. In some embodiments, the outer sections 24a, 26a are bendable downwardly to attach to the operating table and the inner sections 24b, 26b are rollable upwardly for shoulder contact/abutment. In alternate embodiments, the inner sections 24b, 26b are bendable downwardly to attach to the operating table and the outer sections 24a, 26a are rollable upwardly for shoulder contact/abutment. Note the sections 24a, 26a, 24b and 26b can be formed such that they are each independently bendable upwardly or downwardly as decided by the clinician for table securement or patient shoulder contact; in other embodiments the sections 24a, 24b, 26a, 26b can be formed so that one or more of the sections can only be bent upwardly and one or more of the sections can only be bent downwardly. In still other embodiments, one or more of the sections 24a, 24b, 26a. 26b are not bendable and remain in the straight (planar) position. Stated another way, in the embodiment of FIG. 1A, all of the four sections 26a-26d are bendable upwardly or downwardly, however, it is also contemplated that one or more of the sections 26a-26d could be designed to bend only downwardly or only upwardly. It is also contemplated that in some embodiments, less than all the sections 26a-26d are bendable upwardly and/or downwardly. It is also contemplated that the bendable sections 26a-26d are not all bendable independently, but one or more sections are bendable together. Metal strips described below can support (reinforce) the bendable sections 26a-26d to help maintain the sections in the bent position.

In an alternate embodiment, instead of a pair of sections on opposing sides of the head rest 14, a single section is shown on each side of the head rest 14 as depicted in FIGS. 1B and 1C. More specifically, device 10′ of FIGS. 1B and 1C is identical to device 10 of FIG. 1A except instead of two sections 24a, 24b and 26a, 26b on each side of head rest 14, a single section 24a′ is on the left side of head rest 14 and a single section 26a′ is on the right side of the head rest 14, the sections 26a″, 26b″ bendable downwardly for table attachment and/or upwardly for patient support. In all other respects, device 10′ is identical to device 10 of FIG. 1A, e.g., base 12, bendable bottom sections 28a, 28b, etc., and therefore all other features/functions of device 10, including alternatives disclosed herein, are fully applicable to device 10′ and for brevity are not further discussed herein.

FIGS. 1A, 2A and 2B show the sections 24, 26 in the flat parallel (non-bent) position. Each bendable section 24a, 24b, 26a, 26b includes a bendable metal strip 40, embedded (captured) inside the foam, shown in the transparent view of FIG. 2A. The metal strip 40 maintains the respective bendable section 24a, 24b, 26a, 26b in the bent position when bent. In some embodiments, the metal strips 40 are 0.032 inches thick, although other dimensions e.g., thicknesses, are also contemplated. Note that in the absence of such metal supports, the foam material would not stay in the bent position and tend to return toward or to the straight/non-bent position of FIG. 1A. FIG. 1D shows the outer sections 24a, 24b in the downwardly bent position and inner sections 24b, 26b in the rolled position as an example of one use of device 10. Note the metal strips 40 in the illustrated embodiment have a width W1 less than the width W2 of metal strips 42 of extensions 28a, 28b (see FIG. 2B) due to the different widths of sections 24a, 24b, 26a, 26b and extensions 28a, 28b. However, in alternate embodiments, the metal strips 40, 42 can have the same dimensions or different dimensions relative to one another. Note that in alternate embodiments, not all of the sections 24a, 24b, 26a, 26b would have metal strips embedded therein depending on the desired use of the device.

Note the metal strips 40 and 42 in the illustrated embodiments, are positioned inside cutouts in the foam material so they are retained within the foam material. Alternatively, the base 12 can comprise two layers of material with the metal strips positioned between the two layers, and then the two layers secured together, with the metal strips maintained therebetween. The two layers can be the same foam material or the additional layer to sandwich the metal strip between the two layers can be of a different material than the foam material of the other layer. The two layers can have the same thickness or different thicknesses.

The head rest 14, as noted above, in preferred embodiments, is removably attached to the upper surface 12a of the base 12 at the top portion thereof. The head rest 14 forms a cushion for the patient's head. It can be attached to the base 12 by Velcro, although other methods of attachment are also contemplated. The head rest 14 can be positioned at various positions along the longitudinal axis L to adjust for patients of different heights, e.g., for smaller patients, the head rest 14 would be attached closer to the middle portion (midpoint) of the device 10. FIG. 1C illustrates Velcro or Velcro friendly strip 38 on the base for attachment of head rest 14, which has a Velcro strip on its underside to attach to Velcro strip 38. Such Velcro strips (or other attachment methods) can be utilized with the embodiment of FIG. 1A for attaching the head rest. The mounting/attachment strips can be of a different size than shown and more than one strip 38 can be provided. Alternatively, a Velcro friendly covering material can cover a portion of the base 12 in the manner discussed below in the embodiment of FIG. 11, i.e., Version II, to provide a wider surface area for head rest attachment.

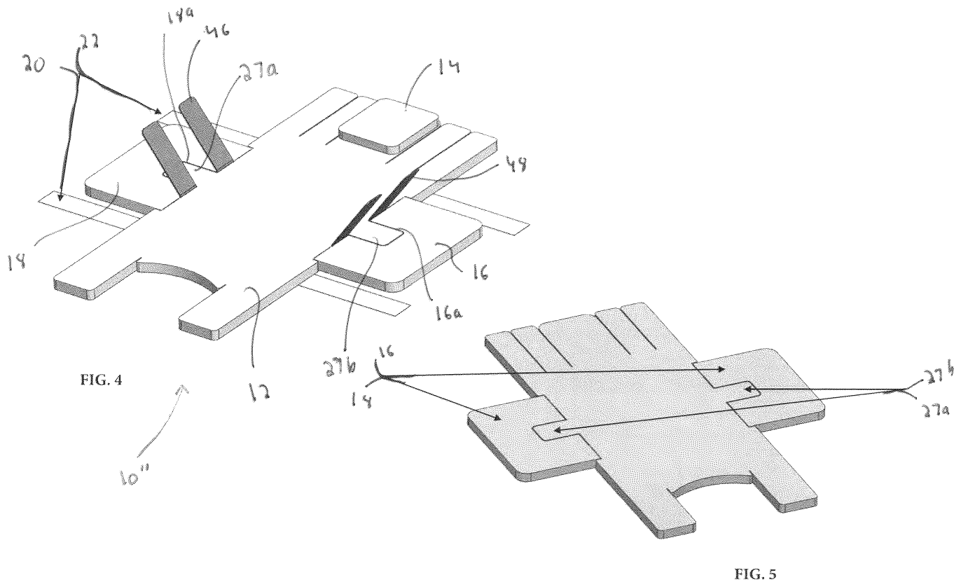

Left and right arm extensions 16 and 18 can be integral with the base 12, however, in alternate embodiments they can be removably attached to the base as shown in FIGS. 4 and 5. More specifically, the extensions 16, 18 of positioning device 10′ include side center extensions 27a, 27b, extending from middle (center) portions of the base 12′ and can be bent upwardly or downwardly. Left and right arm sections/extensions 16, 18 each have Velcro strips 54, 55, respectively (FIG. 3). A metal strip 44 (FIG. 2A), extending transverse to the longitudinal axis L, and embedded in the foam base 12, maintains the side extensions 16, 18 in the bent position out of the plane of the base 12 as it wraps around the bent arm section. The arm sections 16, 18 can be bent upwardly to assist holding the patient on the base 12 or bent downwardly to wrap around the OR table to secure the sides of the base 12 to the operating table. FIG. 1A shows one such configuration which the arm sections 16, 18 can be bent into, although other configurations are also contemplated. Note the arm sections 16, 18 are preferably bent once they are attached to the base 12. The side extensions 27a, 27b of base 12 are each configured to fit within the cutout region of the arm sections 16, 18, respectively, providing an interference fit for attachment for the arm sections to the base.

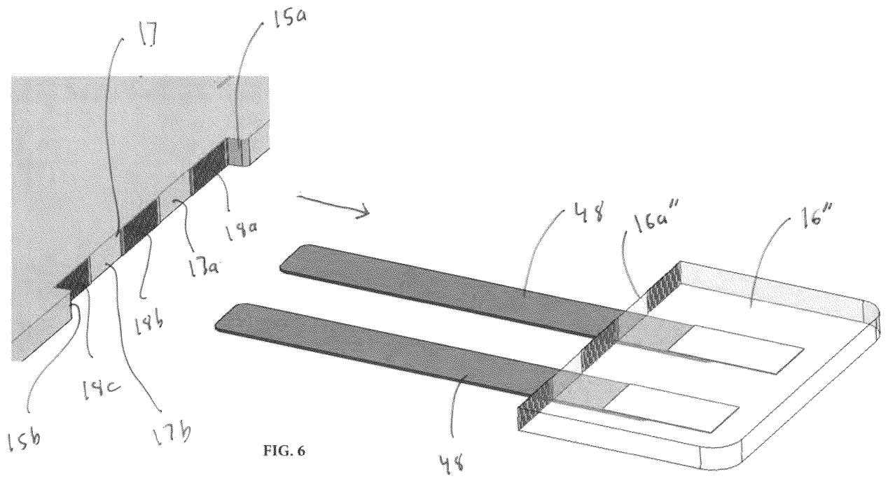

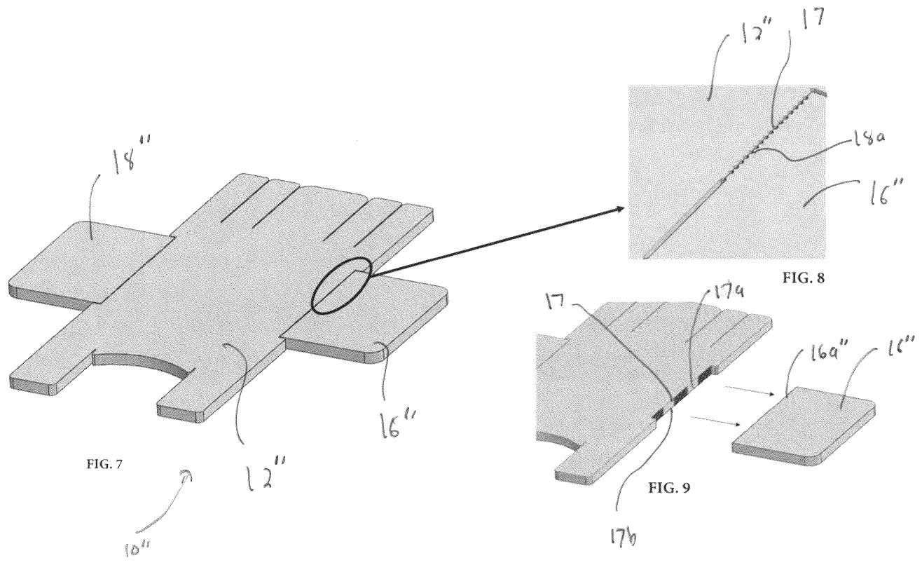

One alternate method of detachment/attachment of the side sections to the base is shown in FIGS. 6 and 7. Side section 16″ is shown in these Figures, it being understood that side section 18″ could be attached/detached in a similar manner. Side section 16″ of device 10″ has a tear apart edge cooperating with the right side edge 17 of base 12″. There are three tear-apart sections shown, designated 18a, 18b and 18c, and collectively referred to as tear apart section 18. The tear apart sections 18a, 18b, 18c are spaced apart to leave a space for the straps 48 which extend through the gap between the inner edge 16a″ of side section 16″ and the outer edge 17 of base 12″. That is, gap 17a between tear apart sections 18a and 18b enables passage of one of the straps 48 through the foam and gap 17b between tear apart sections 18b and 18c enables passage of the other strap 48 through the foam. Note edge 17 forms a slight cavity between walls 15a and 15b dimensioned to create a region for receiving the side section 16″. Preferably the left side section 18″ and left side edge of the base 12″ are the same as the right side section 16″ and right side edge of the base 12″ and engage and detach in the same manner as side section 16.″ Straps 46 would extend through the regions devoid of tear sections in the same manner as straps 48.

Note in the embodiment of FIGS. 4 and 5 which have the extensions 27a, 27b, the side sections 16, 18 in alternate embodiments can have tear away features to separate in the same manner as in FIGS. 6 and 7 or can rely solely on separation of the frictional engagement, i.e., separating the interference fit of the extensions 27a, 27b with the respective side section.

With reference to FIGS. 1A and 2A, the left side section 16 and a right side section 18 which provide arm rest pieces for the patient's left and right arms (and thus also referred to herein as left and right arm sections 16, 18), respectively, and help cradle the patient, are shown in the non-bent planar position in FIG. 2A and the bent position in FIG. 1A. As discussed above, in some embodiments, including the illustrated embodiment, the side sections 16 and 18 are removably attached to the base 12 via an interference fit (with side extensions/side tabs 27a, 27b) or other methods of attachment. Attached to left side section 16 are two straps 48 and attached to right side section 18 are two straps 46. The straps 44, 46 in some embodiments remain with the side sections 16, 18 when the side sections 16, 18 are removed (detached) from the base (as in FIG. 6). Side sections 16 and 18 can be bent upwardly or downwardly with respect to base 12, or can be rolled from a planar configuration (as in FIG. 4) to U-shaped configurations (34, 36) as shown in FIG. 1A. Although two straps for each section 16, 18 are illustrated a single strap or more than two straps are also contemplated.

If the side sections 16, 18 are bent into a U-shape as shown in FIG. 1A, they can be retained by side straps 48 and 46. More specifically, straps 48a, 48b (collectively straps 48) are wrapped around side section 16 to secure the side section 16 in the bent position. The straps 48a, 48b each have a Velcro portion to attach to the Velcro 54 (FIG. 3) on the underside of section 16. The straps 48 extend in a cutout or space between the base 12 and side section 16 and thus can come up from an underside of the side section 16, through a gap between the side section 16 and side edge of the base, and then wrap around the bent section 16 to attach to the Velcro section 54. Similarly, straps 46a, 46b (collectively straps 46) are wrapped around side section 18 to secure the side section 18 in the bent position. The straps 46 each have a Velcro portion to attach to the Velcro 55 (FIG. 3) on the underside of section 18. The straps 48 extend in a cutout or space between the base 12 and adjacent side section 18 and thus can come up from an underside of the side section 18, through a gap between the side section 18 and adjacent side edge of the base and then wrap around the bent section 18 to attach to the Velcro section 55.

Device 10 further includes transversely extending bed straps 20 and 22 which are connected to a lower surface of the base 12 and are used to attach to the bottom of the operating table, e.g. wrap around the metal rails. These straps 20, 22 can include a Velcro portion to which one end of the strap attaches when it is wrapped around the table so they attach to themselves. Other forms of attachment are also contemplated. The straps 20, and 22 can also be used in the opposite direction i.e., wrapped around the patient positioned on the base 12 and can be secured by Velcro or other attachment structure so they attach to themselves. Although two straps 20, 22 are illustrated, a single strap or more than two straps are also contemplated for wrapping around the operating table and/or patient, and the straps can be placed in positions other than those shown.

In some embodiments, hydrogel pads 50 and 52 attached to the underside of the base 12 (FIG. 3) can be provided to add to the coefficient of friction of the base 12 to reduce sliding of the positioning device 10 on the operating tables as the pads 50, 52 lie atop and in contact with the table 12. A different number of hydrogel pads is also contemplated. The hydrogel pads can also be provided on the top surface of the positioning device 10 in direct contact with the skin to provide skin protection. The hydrogel pads 50 and 52, as well as the aforedescribed straps 20, 22 can be used with the other positioning device embodiments disclosed herein.

The base pad in some embodiments can include slits formed therein. Such spaced apart slits can be provided in select regions of the pad to contour to get it to arc better. These select cuts allow select regional adjustments to the base foam compliance. For example, slits in the lumbar region would allow more compliance (ability to conform to shape and reduce pressure regions) compared to regions without slits. Thus, the slits provide different zones to provide different compliance to provide different response to loads. The slits can also aid in bending of the base pad.

The bases/pads of the present invention can be asymmetrical. Alternatively, they can be left/right symmetrical as in the embodiment of FIG. 1 or fully symmetrical (left/right and top bottom) as in the embodiment of FIG. 11 discussed below.

FIGS. 11-16 illustrate an alternate embodiment of the device of the present invention. This device as referenced above is the modular attachment version—Version II. This device differs from the device in FIG. 1 in that a) it has a Velcro friendly (also referred to herein as Velcro compatible) covering material over the base/pad (or alternatively, a Velcro friendly based/pad material) to enable attachment of supports anywhere on the base and is not limited by the location of the specific placement of the pre-attached Velcro strips; b) adjustable supports-supports of various shapes and sizes can be removably attached throughout the pad (via the Velcro friendly cover or material); c) a metal support extends along a length and width of the foam pad to provide attachments to the operating table/bed; and; d) it has a full symmetric design so the pad can be used in either orientation. Note that the positioning devices of the present invention can have all of the foregoing features a-d, or fewer than all such as one or two of these features, and still provide advantages over current patient positioning devices.

Also, in some embodiments, the positioning device has a double layered pad design as described in detail below which adds additional symmetry by enabling the device to be used effectively on either side, as described below.

The devices of FIG. 11-16 and FIGS. 17A-30 include a Velcro-friendly/compatible cover/layer/material overlying or integrated with the pad to mount various supports to the upper surface.

Note the term Velcro as used herein denotes the name of the hook-and-loop fastening system which provides an adjustable closure as the hooks catch the loops to create a strong bond as they temporarily attach (stick) to each other. That is, the two layers comprise a hook side having a fabric covered with plastic hooks and a loop side comprising plastic loops adapted to engage with the hook side. Velcro can be made of a variety of materials such as nylon, polyester, polypropylene, stainless steel, etc. A “Velcro friendly” or “Velcro-compatible” or “hook-and-loop fastener-friendly” or “hook-and-loop fastener-compatible” material as used herein denotes either Velcro itself or a material in which Velcro can attach to such as a fabric like cotton, nylon or polyester. Thus, the fabric can provide patient comfort while adhering Velcro strips/Velcro material thereto. It provides a surface safe for the patient's skin.

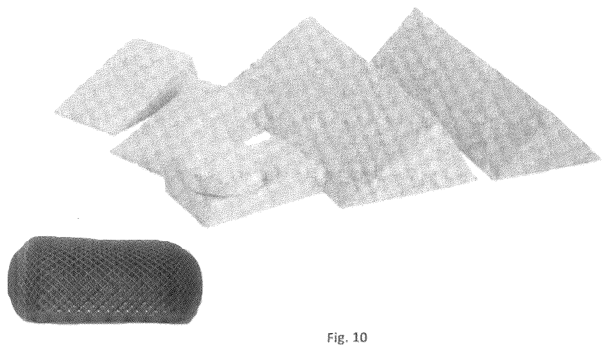

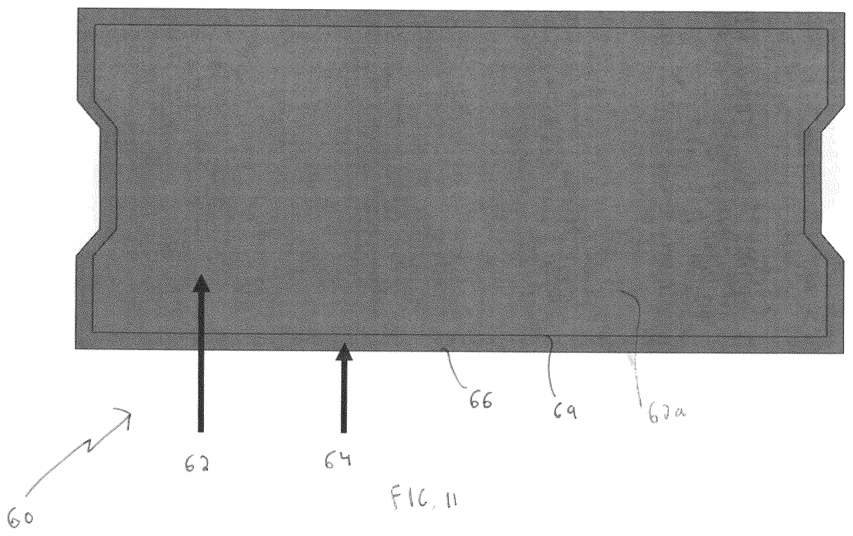

Turning now to the embodiment of FIGS. 11-16, and with initial reference to FIGS. 11 and 12, the device, designated generally by reference numeral 60, has a pad 62, preferably composed of foam like pad 12 of FIG. 1, (although other materials are contemplated) which is covered or substantially covered by a Velcro friendly material 64 to provide a Velcro attachment surface and thus allow for versatile attachment options. This provides a “stick anywhere” feature to increase usability across various settings. In the foregoing embodiments (Version I), the pad included Velcro strips at select regions for attachment of the supports or modules, e.g., head rest and arm extensions, and could further include other Velcro strips (not shown) at the appropriate region for attachment of other supports or modules such as lumbar support, shoulder support, shoulder stop, back stop, etc. However, the Velcro friendly material of device 60 of the embodiment of FIG. 11 enables the supports or modules, such as those shown in FIG. 10, e.g., shoulder, head, neck, lumbar, etc., to be attached anywhere on the base/pad and thus are not limited by the location of the specific attached Velcro strips as for example in the embodiment of FIGS. 2A and 2B discussed above. This “stick anywhere” feature also allows for the patient and/or bed rail straps to be attached anywhere on the base/pad.

As shown, the material 64 covers the entire top surface of the base 62, and can wrap around the peripheral edge 66 of base 62 forming a lip 69 extending around the periphery (partially or alternatively around the entire periphery as shown in the illustrated embodiment) for better securement. Note alternatively the material can wrap around the bottom surface 62a of base 62 to greater extent than shown and in some embodiments can wrap around the entire base (the entire top and bottom surface) to fully encapsulate the base 62. In the illustrated embodiment, since the cover 64 only wraps around the edge, bottom surface 62a is exposed. In alternate embodiments, the material 64 covers only a portion, e.g., a majority portion of the top surface, but not the entire portion of the top surface of the base. The cover material 64 can be integrated with the pad or attached to the pad by adhesive, laminated, sewn into the foam material or by other attachment methods. The cover material also provides a smooth non-irritating skin-friendly interface with the patient so as not to cause patient skin irritation or rash for patient safety and comfort. (The supports disclosed herein can also provide a smooth skin-friendly interface with the patient).

The covering can in some embodiments comprise an integrated aggressive micro (super fine) Velcro with high strength for secure attachments without adding bulk. This allows for adjustable configurations while maintaining a sleek design.

With the use of the Velcro friendly covering/material and mounted supports, different foam materials can be used than in current foam pads. For example, a less expensive foam such as a polyurethane foam, rather than a memory foam, can be utilized, although other materials are also contemplated. That is, without the mounted supports of the present invention, a more expensive memory foam might need to be used to conform to the patient's anatomy to secure the patient and limit sliding.

The covering material can also enable incorporation of a logo or other indicia into the material to identify the device, manufacturer, or provide other information.

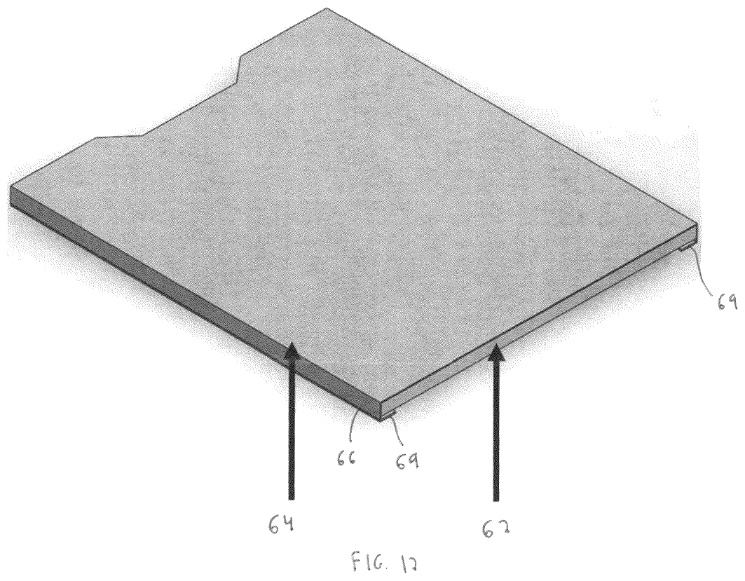

The device 60 further includes a metal support or splint 70 extending along a substantial length and/or width. The support 70 can be in shape of an H as shown in the bottom view of FIG. 13, the shape of a Figure eight, an X, or other shapes to extend along the pad. With the H-shape, metal support portion 72 extends along a length, i.e., along a longitudinal axis of the base 62, and has two axially spaced apart metal support portions 74, 76 extending transversely to the longitudinal portion 72 and laterally pas the left and right side edges of the base 62. Support portions 74, 76 can be at 90° angles to the longitudinal portion or at other angles. Note the metal support 70 can be composed of a single piece of metal or alternatively composed of multiple pieces of metal attached together, e.g., supports 74, 76 attached to support 72, support 74 composed of two or more pieces attached together, etc. The metal 70 is bendable out of the plane so it can be used to wrap around the bar or rail of the operating table to provide rigid fixation to the table. The metal support 70 is shown in the non-bent planar position in FIGS. 13 and 14. It can be attached to a bottom surface of base 62 or positioned within the foam pad. Note in alternate embodiments, only one transverse support portion could be provided or more than two transverse support portions can be provided axially spaced along the longitudinal axis. The longitudinal support 72 and transverse supports 74, 76 can extend for a longer or shorter length than shown in FIG. 13. In alternate embodiments, support portions 74, 76 can wrap around the patient.

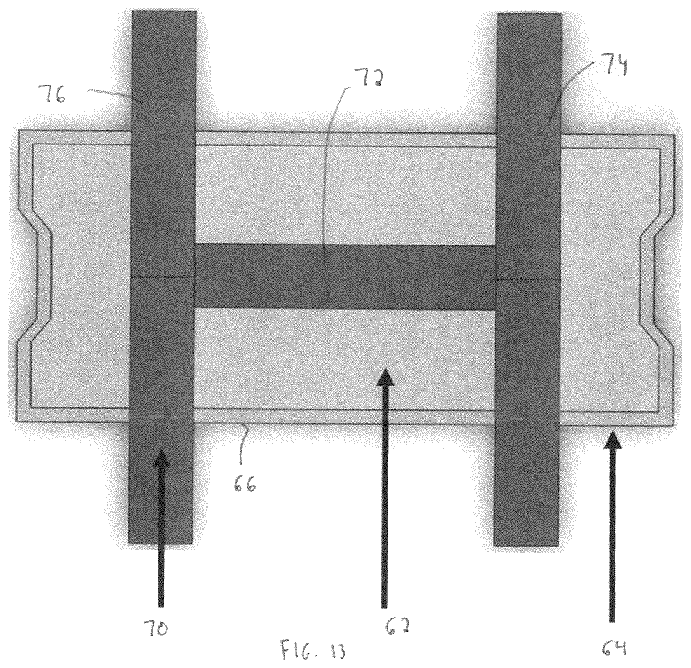

The ends of the two metal supports 74, 76 extend beyond the periphery 65 of the pad 62 and are of a sufficient length so they can be bent around the table. The extending portions 74a, 74b and 76a, 76b are shown exposed in FIG. 15A but in some embodiments, extending portions 74a, 74b are covered by foam extensions 62a, 62b, respectively, and/or extending portions 76a, 76b are covered by foam extensions 63a, 63b, respectively, as shown in FIG. 15B. One or more extending portions 74a, 74b, 76a, 76b can alternatively be covered by other protective material such as by a rubber spray. One advantage of the metal is that it doesn't twist in the same manner as other material such as Velcro (which can tear) if used as a tension strap. With the metal in the embodiment of FIG. 13 running continuously through the foam pad 62, e.g., through a center of the foam, it can also assist lifting the patient from the surgical table to a transport device for moving the patient.

The metal support can be covered with foam as described above and shown in FIG. 15B or alternatively can be encapsulated or covered in another protective material of a soft membrane to cover any sharp edges. Metals that can be used for example are aluminum (which is radiolucent), copper, stainless steel, etc. The metal selected needs to strike the balance of being sufficiently thick to provide sufficient clamping/retention force on the table when bent around the rail but not too thick which could limit its bendability. The material and thickness of the metal need to strike a balance between these two competing factors.

The pad 62 is shown as a symmetrical design. One advantage of the symmetrical foam pad 62 is that the user does not need to worry about the positioning of the device on the medical table nor the patient's positioning since the “top” and “bottom” are identical and the “right” and “left” are identical. Thus, for example, the head rest can be attached on either the “top” or “bottom” of the foam pad.

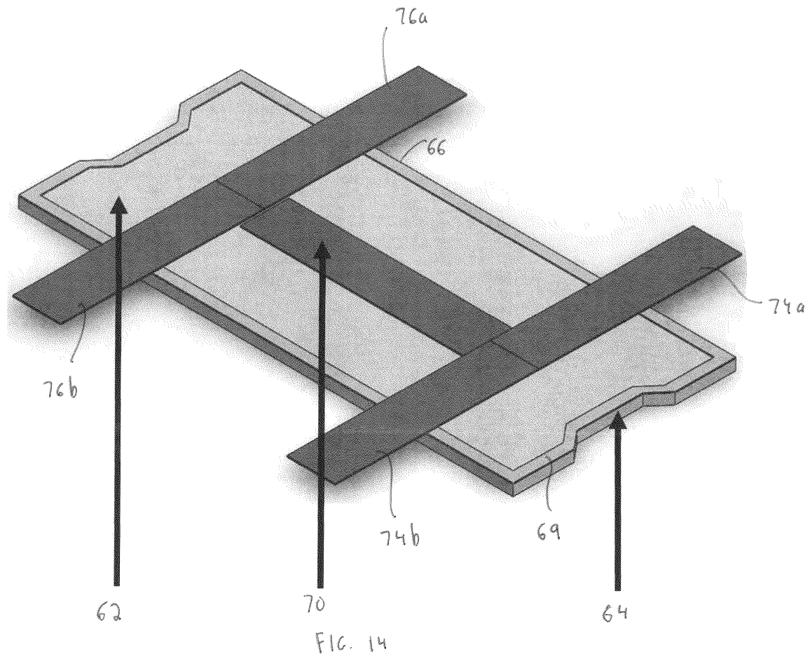

To provide further symmetry, a second pad layer, preferably composed of foam although other materials are also contemplated, can be placed over the first layer as shown in the embodiment of FIG. 16. This provides additional symmetry so the device can be used on either side. As shown, in this double pad design, the metal support 70 is sandwiched between the first foam pad 62 and the second foam pad 78. Foam pad 78 contains or is covered by a Velcro-friendly material 79 in the same manner as foam pad 62, thus forming a four-layer design-material 64, pad 62, pad 78, and material 79. The Velcro-friendly material 79, like Velcro-friendly material 64, can partially or fully encapsulate the foam pad 78 and partially or fully cover the “support receiving” surface and provides a surface for attachment of the support or modules in the same manner as foam pad 62. In this manner, the device can be used with either covering material 64 or covering material 79 facing the patient with the other material facing the table. Note the discussion above of foam pad 62 and covering material 64, and their alternatives, are fully applicable to foam pad 78 and covering material 79. Pads 62 and 78 can be composed of the same material or a different material. Instead of covering material, the base/pad itself can include a Velcro friendly material.

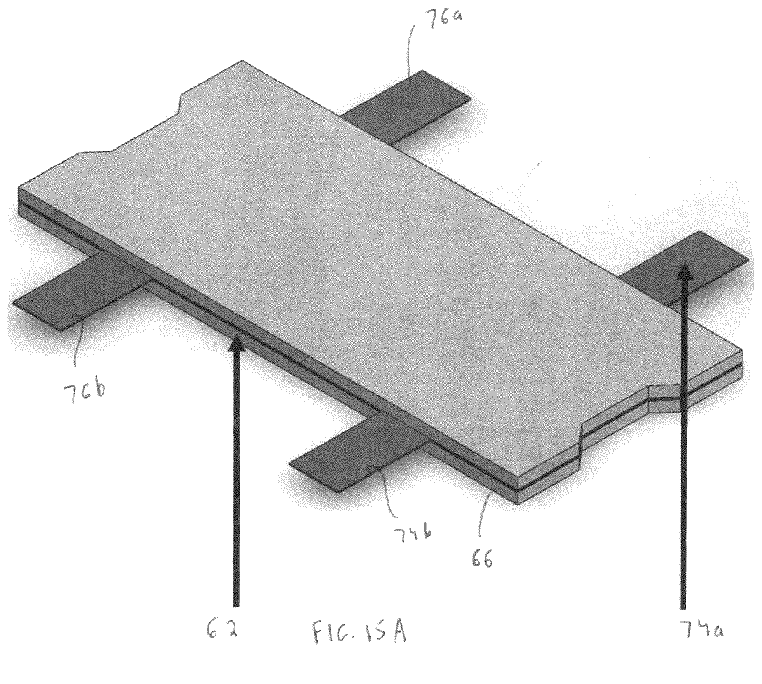

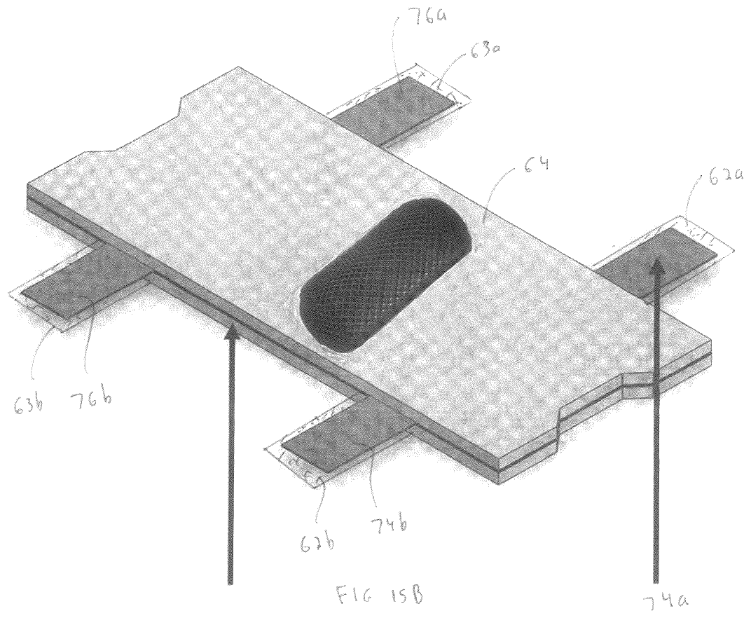

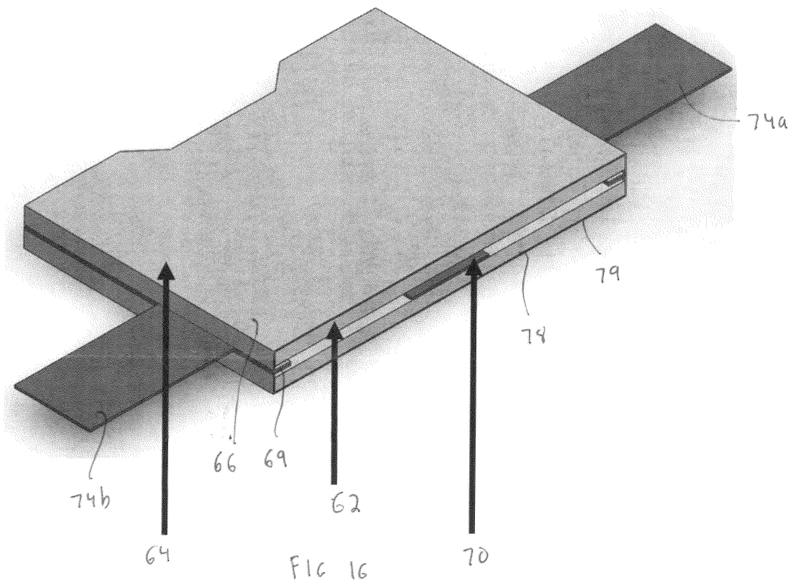

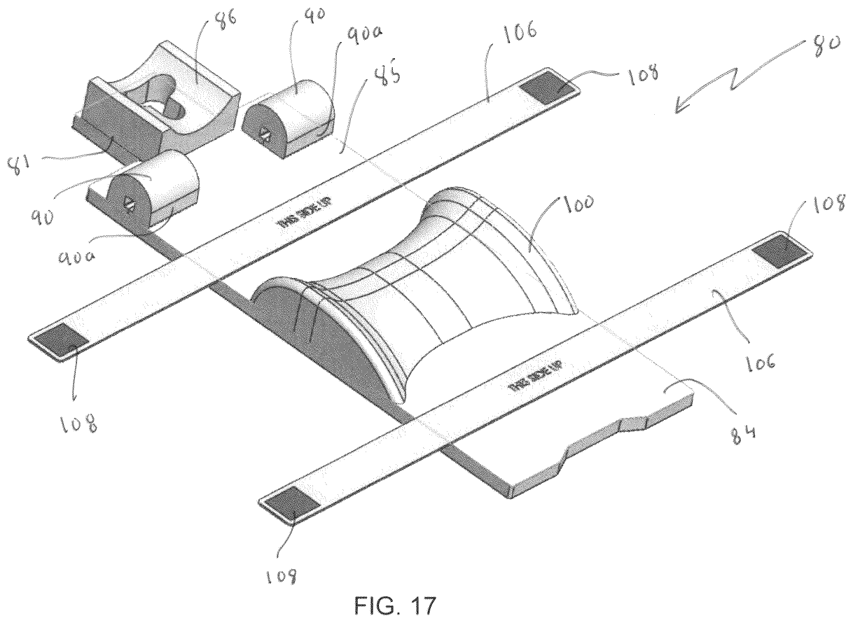

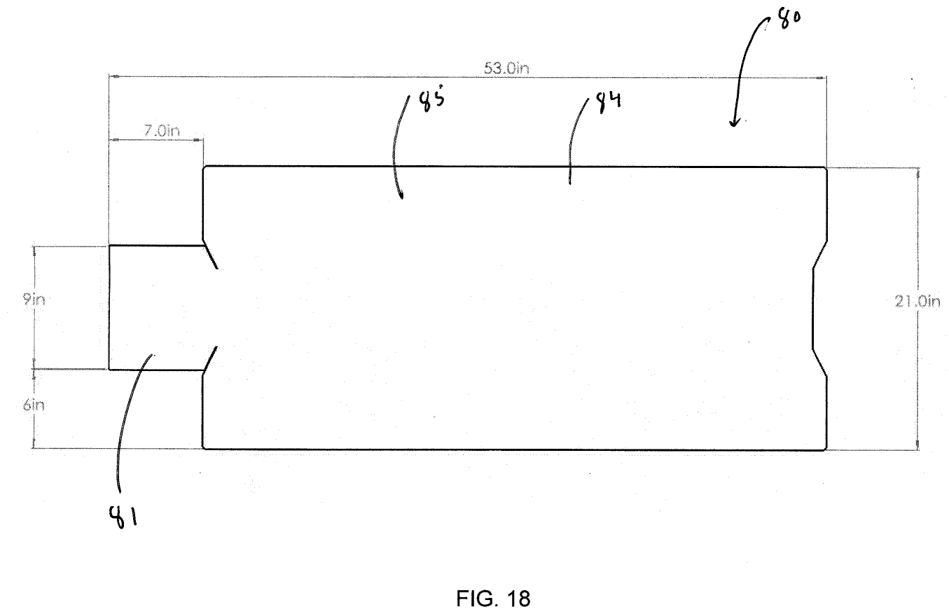

FIG. 17 illustrates an alternate embodiment of the patient positioning device of the present invention, which is an alternate embodiment of the modular attachment version—Version II. Patient positioning device 80 is similar to the patient positioning device 60 of FIG. 11 except as discussed herein. Positioning device 80, has a head extension 81, as shown in FIG. 18. This can receive a head rest 86. Patient positioning device 80 has a Velcro-friendly material 84 on the pad 82 to provide an adherence surface on upper surface 85 for mounting various supports. The Velcro-friendly material 84 can be a cover over at least a top surface of the pad, a top layer attached to the pad such as by adhesive or laminated, or integrated with the pad, and can be a micro-Velcro. Material 84 functions in the same “support mounting” manner as cover 64 described above and therefore the material 84 and its features and benefits for brevity are not repeated herein. The above description of the Velcro-friendly material, and its alternative size, materials, etc., is fully applicable to the material 84 of patient positioning device 80. Material 84 is selected to reduce patient skin irritation.

The covering material 84 can also enable incorporation of a logo or other indicia into the material to identify the device, manufacturer, or provide other information.

The pad can be composed of a non-memory biocompatible foam, and composed for example from polyurethane, with a Velcro friendly interface, composed for example of polyester, to provide a smooth surface that doesn't stick to the patient's skin and doesn't irritate the patient's skin. Thus, the patient interface is a smooth material which Velcro strips can adhere to. The Velcro friendly surface can be a covering material that is placed over the pad or a layer of material integrated with the pad during manufacture to form an upper base covering. It can be formed, e.g., integrated with the pad, for example by a fabric sheet laminated or glued over an upper surface of the pad.

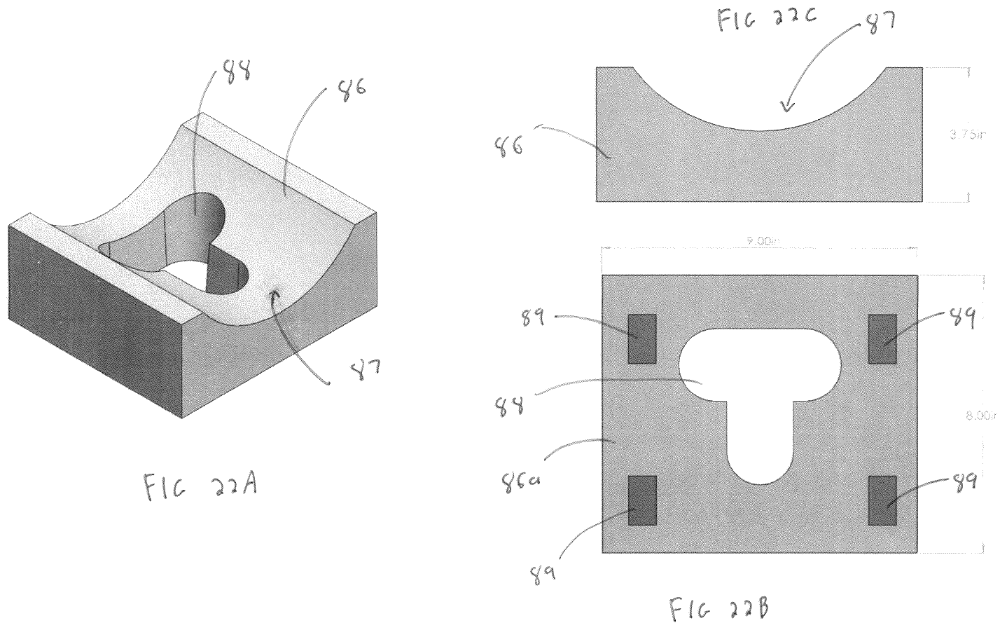

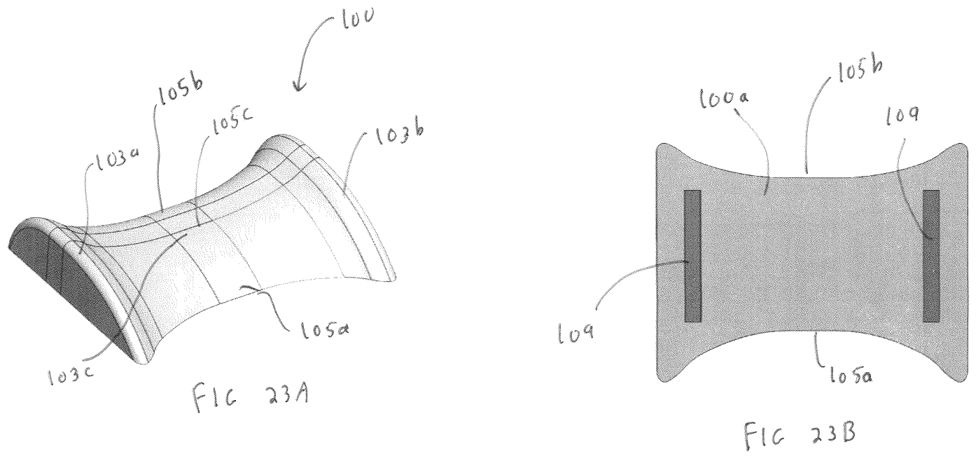

A head rest 86 is shown attached to head extension 81. Head rest 86 can be removably attached to upper (top) surface 85 and is preferably placed on the head extension area 81 to support the patient's head. The head rest 86, with reference to FIGS. 22A-22C, has an open configuration and forms a concave cradle 87 to comfortably receive the head. A cutout 88 provides comfort in a face-down position. On the lower surface 86a of head rest 86, four Velcro strips 89 are provided, arranged around the periphery around the cutout 88, to adhere to the Velcro friendly material 84. The number, size and location of the Velcro strips can differ from that show in in FIG. 22C. In some embodiments, the head rest 86 is composed of a foam material.

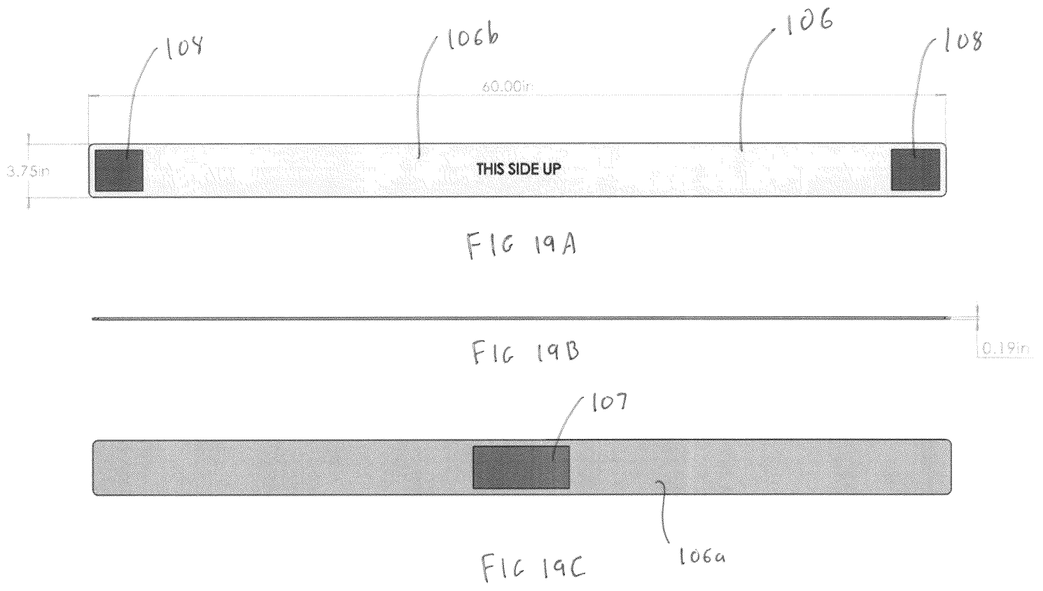

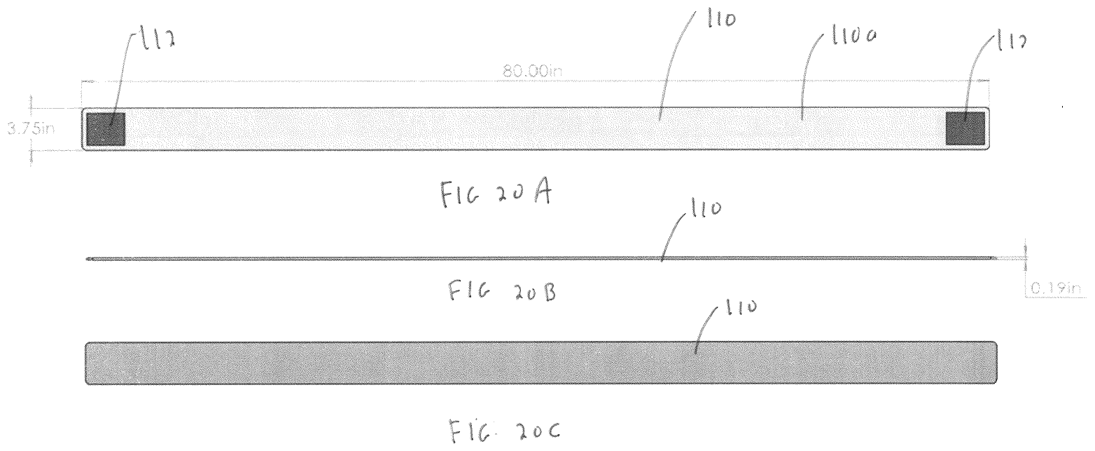

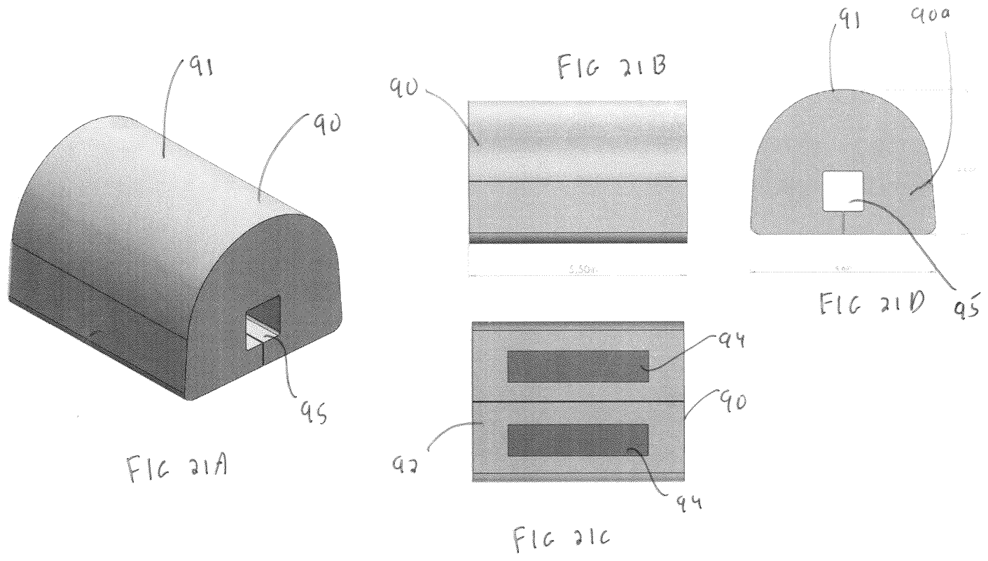

A shoulder bump (support) 90 is provided near an upper top portion of pad 82, one on each side of the device 80. Each shoulder bump 90, with reference to FIGS. 21A-21D, has an arcuate/arch shaped upper wall 91 (forming a U-shape). Contact surfaces 90a on a front side restrict movement of the patient. On the lower surface 92 of shoulder support 90, two Velcro strips 4 are provided, to adhere to the Velcro friendly material. The number, size and location of the Velcro strips can differ from that shown in in FIG. 21C. A channel or opening 95 extends through the shoulder support 90. The shoulder bumps can be removably attached to surface 85.

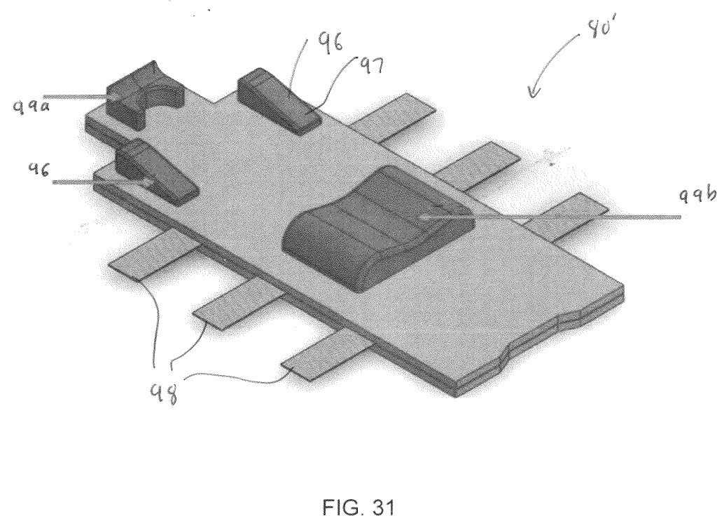

In the alternate embodiment of FIG. 31, shoulder bumps 96 are substantially triangular cross section and have an upper surface 97 tapering in a direction away from the head. The head rest 99a and lumbar support 99b also differ in configuration from head rest 86 and lumbar support 100. The positioning device 80′ has a series of bed straps 98 similar to straps 20 described above for wrapping around the medical table. In all other respects, the positioning device 80′ is the same as positioning device 80 and for brevity will not be further described herein.

A lumbar support 100 is shown attached to upper surface 85 of Velcro-friendly material 84. Lumbar support 100 can be removably attached to surface 85 and is preferably placed where arc is in the spine of the patient. Lumbar support 100, as shown as FIGS. 23A and 23B, has left end region 103a and right end region 103b. Intermediate region 103c is between the end regions 103a, 103b and has a curved upper surface on which the patient's lower back can rest. The intermediate region 103c is recessed relatively to the end regions 103a, 103b. Stated another away, the end regions 103a, 103b extend a greater distance from the upper surface 85 of Velcro-friendly material 84 than intermediate regions 103c, thereby forming lateral raised regions and creating an indentation to limit side to side (lateral roll) movement of the patient, especially if the medical table is rotated at an angle to the floor. Lumbar support intermediate region 103c can have a convex surface for patient comfort and support. Front and back curved surfaces are designated by reference numerals 105a, 105b, and the upper wall 105c of the lumbar support 100 curves downwardly from the center of intermediate section 103c to the outer wall 105a and to the outer wall 105b.

On the lower surface 100a of lumbar support 100, two Velcro strips 109 are provided, to adhere to the Velcro friendly material 84. The number, size, shape and location of the Velcro strips can differ from that shown in FIG. 23B.

Although described as strips, other non-strip configurations are contemplated for attaching the various supports to the pad.

Note lumbar support 100, instead of being one piece (component), can comprise two or more components placed side by side to provide patient lumbar support. The lumbar support 100 helps to prevent side-to side rolling of the patient.

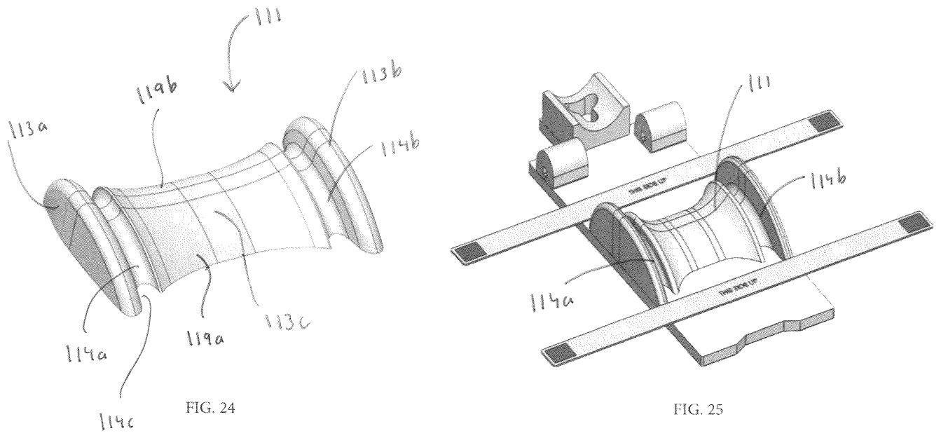

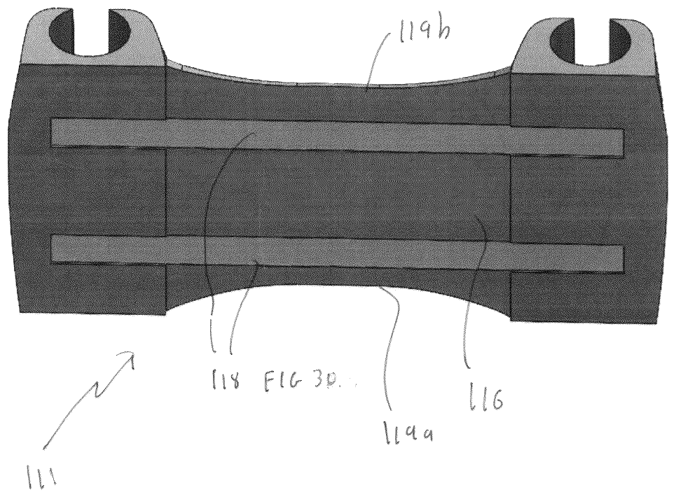

In the alternate embodiment of FIGS. 24-25, lumbar support 111 has a pair of arm slots channels—right arm channel 114a is formed in the right-side region 113a of lumbar support 110 and left arm channel 114b is formed on the left-side region 113b of the lumbar support 111. Intermediate region 113c, like intermediate region 105c of FIG. 23A, is between the end regions 113a and 113b and has a curved upper surface on which the patient's lower back can rest. The channels 114a, 114b capture the arms to secure the arms without restricting blood flow. The visualization channels 114a, 114b also provide for constant arm visualization such as for constant direct visualization of the IV site. The intermediate region 113c is recessed relatively to the end regions 113a, 113b. Front and back curved surfaces 119a, 119b are provided and the upper wall of the lumbar support 110 curves downwardly from the center of intermediate section 113c to the outer wall 119a and to the outer wall 119b. On the lower surface 116 (FIG. 30) of lumbar support 111, two Velcro strips 118 are provided to adhere to the Velcro friendly material 84. The number, size, shape and location of the Velcro strips can differ from that shown in FIG. 30. Lumbar support 111 prevents side-to-side rolling and helps secure the patient's arms in place, e.g., locks the patient's arms in position. This dual functionality improves stability and user comfort. Thus, the mobile lumbar support 111 has high skin adhesion and mechanical support to provide additional comfort and positioning options for users. Note lumbar support 111, instead of being one piece (component), can comprise two or more components placed side by side to provide patient lumbar support. In a multiple component support, one arm channel could be in one component and the other arm channel in a second (another) component.

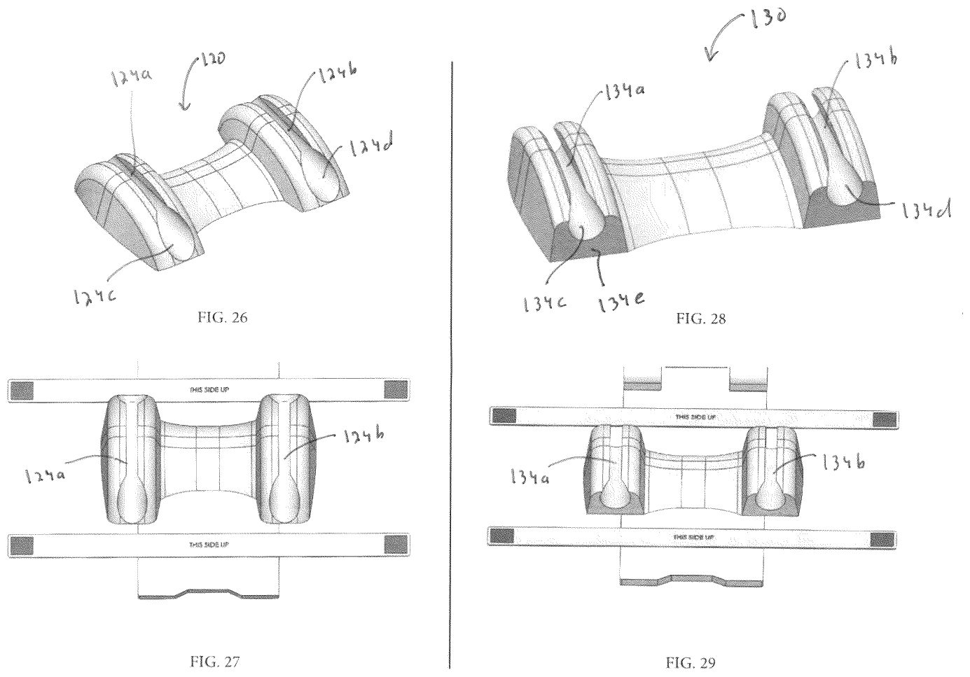

FIGS. 26-29 illustrate two alternate embodiments of the arm slots/channels. Arm channels (slots/tunnels) 124a and 124b of lumbar support 120 of FIGS. 26 and 27 have a teardrop shape 124c, 124d, respectively, to provide a widened area in the front region. Arm channels (slots/tunnels) 134a and 134b of lumbar support 130 have a widened area 134c, 134d, respectively, in the front region forming a more cone-like shape. Widened areas could also be provided in the rear region. In all other features, lumbar supports 120 and 130 are identical to lumbar support 111 so for brevity, the description of such featured, and their alternatives, which are fully applicable to lumbar supports 120, 130, are not repeated herein.

Channels 124a, 124b 134a, 134b also differ from channels 114a and 114b since they terminate in the lateral region and do not extend to open at the lowermost wall of the lateral region (compare region 134e of FIG. 28 and region 114c of FIG. 24).

Note the lumbar supports 120 and 130 of FIGS. 26-28 also differ from lumbar support 100 of FIG. 17 in that they extend laterally beyond the right and left edges of the pad in order to better accommodate the arms of the patient lying on the pad which can be positioned outside the width of the pad.

Note the lumbar, shoulder and head supports, as well as the bed straps and patient straps, can be used with the other embodiments of the patient positioning devices disclosed herein.

Referring back to FIG. 17, and with reference also to FIGS. 19A-19C, straps 106 loop through or under the rails of the medical table to attach to itself. Each strap 106 has a Velcro adhering surface 107 in a central region of bottom surface 106a for attachment to the Velcro friendly material 84 at a desired region, thus providing an “attach anywhere” strap that can be positioned as needed for each surgical setup. The straps lie on top of the pad and the patient lies on top of the strap. The straps are separate and not integrated or permanently mounted to any specific location which provides versatility since they can be attached at any region to the pad and thus to the table. These movable straps 106 each have a Velcro adhering surface 108 on each end of an upper surface 106b so that when the straps 106 are wrapped, the ends attach together. That is, each strap 106 loops though the bedrails and attaches to itself. The bed straps are designed to accommodate variable table rail configurations and avoids the limitations of pre-paced or sewn-in straps, allowing direct attachment to rails located in positions that may vary between different table models and procedures. The Velcro ends and strips 107 can be micro-Velcro laminated to the strap. The straps 106 preferably have a unidirectional stretch to provide secure attachment while allowing some flexibility in fit. This unidirectional strap enables providing a strip to fit multiple sizes—one size fits all Indicia such as “this side up” can be provided on the upper surface 106b to aid the user. Two straps 106 are shown in the embodiment of FIG. 17, one on each side of the lumbar support, extending transversely across the base and laterally beyond the periphery of the base, although different locations are also contemplated as well as a different number of straps 106. Note in the embodiment of FIG. 17, the straps 106 are attached to the material 84 on an upper surface 85 thereof so the patient lies on an upper surface 107 of straps 106. This further engages the pad to the table and prevents foam pulling away from foam. By providing the wide straps 106 on top surface 85 of the base in the FIG. 17 embodiment, rather than being sewn in, the foam can be compressed (e.g., compressed to the table) and the foam is not weakened in the sewn regions. In alternate embodiments, the straps can be under the pad or within the pad. These straps can in some embodiments also be used as patient straps, wrapping in the opposite direction over the patient as described herein.

Note the straps 106 in preferred embodiments, are designed to extend over the pad of the present invention, e.g., one inch thick, plus the thick pad on the medical table, e.g. 3 inches thick. The straps 106 provide a compressive force to these pads which reduces the chances of the foam ripping.

Patient straps can also be provided. As shown in FIGS. 20A-20C, patient straps 110 have Velcro attachment regions 112, 112 at each end, on an upper surface 110a, for adherence once the strap is wrapped around the patient. In some embodiments, one strap 110 can wrap around the lower pelvis and one strap 110 can wrap around the shoulder. The straps 110 can have stretchability so as not to inhibit breathing. The straps preferably flex in one direction, e.g., in a strap 4 inches in width, when pulled it can stretch to 2 inches in width. The straps 110, like straps 106, can be made of woven material to provide the stretchability. Each strap 110 can attach to itself, via the Velcro ends 112 or alternatively attach to rails of the table. The Velcro ends (as well as the other Velcro strips and surfaces of other supports), can be micro-Velcro and can be laminated or otherwise attached to the component.

The patient and/or bed straps 106, 110 can be provided separately so the user can decide the positioning of the straps. Indicia of “this side up” can be imprinted on the straps.

In some embodiments, a universal strap can be provided to wrap around the rails or patient. Thus, the strap can wrap in either direction.

In some embodiments, to minimize tearing of foam at high stress pints, radial relief cuts are provided and/or non-woven backing material is applied to distribute forces and eliminate tears.

Note the supports can provide barriers to right and left movement as well as front to back movement.

Devices of the present invention can provide a surface that is compliant, skin-friendly and allows direct attachment of straps and accessories anywhere on the device.

Sections of the devices of the present invention can be absorbent or superabsorbent to absorb blood and/or other body fluids. This absorbability can be part of the foam material itself and/or in form of absorbable inserts placed in and/or on the foam. Alternatively, or in addition, wound care materials, e.g., multi-layer wound dressing, providing a wicking agent and an absorbent can be placed on select regions of the foam or on select regions of the covering material in the embodiments utilizing the Velcro-friendly material, such as in the perineum or sacrum area for example. In some embodiments, a section dressing material can be attached, e.g., by adhesive or placed, e.g., wrapped, over a section of the foam or covering material to provide a high-water absorption rate.

The devices of the present invention can include a lumbar support providing a raised support on the pad. In some embodiments, the lumbar support could have a fluid containment feature, such as pockets, to absorb blood and/or other body fluids.

The positioning devices of the present invention advantageously provide fast setup, e.g., 15 seconds or less, via the bendable sections and/or straps.

The positioning devices in some embodiments provide a universal shape. In such embodiments, instead of relying on fixed shapes to position body extremities during surgery, the bendable integrated segment or segments allow for on demand shape contouring. Specifically, the malleable material holds and overcomes deformation loads such that the new deformed shape can support body loads in any geometry, providing a one size fits all technology. Standard and complex shapes, asymmetrical or symmetrical, can be created using the bendable internal segment to hold the shape.

Examples of support or modules are shown in FIGS. 10, 17 and 21A-31, which include for example shapes that are right-triangular in cross-section, equilateral-triangular in cross-section, circular in cross section, rectangular in cross-section, oval in cross-section (lumbar support), etc. These patient supports can be part of the pad but in preferred embodiments are separate connectable components permanently or removably attached to the pad or covering material in the embodiments utilizing the Velcro-friendly covering material or Velcro strips so they can be attached at various locations and orientations. The supports can be packaged together or separately from the pad(s). In some embodiments, the supports can be permanently attached.

The bendable metal segment(s) in some embodiments enables the flat sheet of foam to morph into different shapes such as triangles, rectangles, domes, etc. and hold the shape. It is also possible in some embodiments to cut the foam and bendable segment(s) to various shapes to support various body parts. For example, the sheet of bendable foam could be bent into a box and hold its shape to support the knee, bent into a pyramidal shape, etc.

Although the positioning devices are described for use with an operating table to overlie the operating table or bed, they can also be used to support patients on other patient supporting structure. Thus, the term “patient supporting structure” as used herein is not limited to operating/surgical tables.

Note the supports described herein can be attached/attachable directly to the base or attached/attachable to the base via a covering material interposed between the base and supports. In either case, the supports are considered herein to be “attached/attachable to the base.” They are preferably removably attachable. The supports can be customizable in size and location for various patient anatomies and surgical needs, improving both restrain and comfort.

The devices in some embodiments are packaged sterile; in other embodiments they are packaged non-sterile.

In some embodiments, the devices can be vacuum packaged to reduce storage and shipping size (and cost) such as by way of example 50% or 66%, or by other sizes/percentages. In such embodiments, the permeable bag is first subject to the sterilization agent (ETO), and subsequently (after the sterilization cycle has been completed) a second vacuum bag is used to compress the foam using atmospheric pressure without violating the sterile barrier for reduced size vacuum packaging. In an alternate embodiment, the device is mechanically compressed prior to sterilization, and then ETO sterilized in a compressed (reduced size) state. In some embodiments, the vacuum compression and/or mechanical compression can reduce the size for shipping by about a 3 to 1 ratio. In other embodiments, it can reduce the size for shipping by or more than about a 2 to 1 ratio.

The vacuum-sealed packaging provides space savings and can be provided in sterile and non-sterile versions. It improves storage efficiency and can potentially extend shelf-life.

In some embodiments, the device can include a purple (or other color) colorant/tint to reduce/resist UV yellowing while maintaining a white appearance. This can help prevent discoloration over time without compromising the desired aesthetic.

The devices of the present invention can be designed for single use (disposable). In other embodiments, they can be designed for multiple uses after sterilization between each use (reusable). In embodiments, wherein the pad has a cover, the pad can be reusable and the cover can be disposable. In such embodiments, the cover can be removed and a new sterile cover can be placed over the pad. In other embodiments, with a covering material, multi-layers of covers can be provided, so that after each use, the topmost cover is removed leaving a clean sterile cover exposed for use. In such embodiments, the covers would preferably cover the entire pad (top, bottom, sides) to maintain the underlying pad portion sterile.

In alternate embodiments, the device can be attached to the medical table by wrapping it with a material such as Coflex. In such embodiments, instead of the bendable metal or foam wrapping around the table, such bendable regions need not be provided in the device and attachment can rely solely on the wrapping the material one or more times around the device and table. This could provide an easier to manufacture and less costly positioning device.

The skin coat on the foam eliminates foam pores which reduces or prevents absorption of fluids into the foam.

In some embodiments, the pad can be disposable and designed for use with a single patient.

### Knee Distraction Application of Support

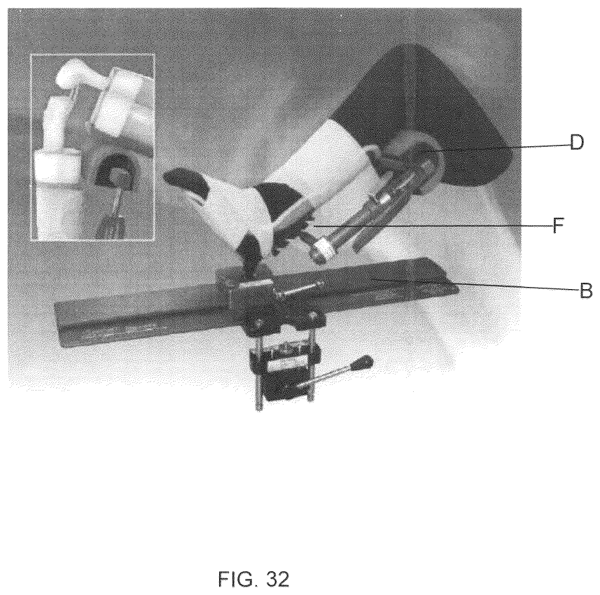

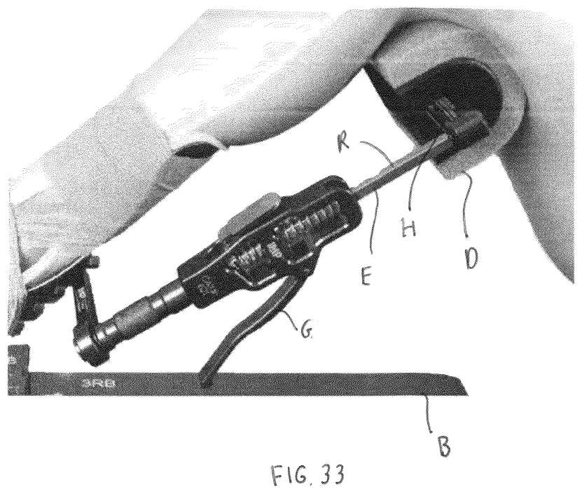

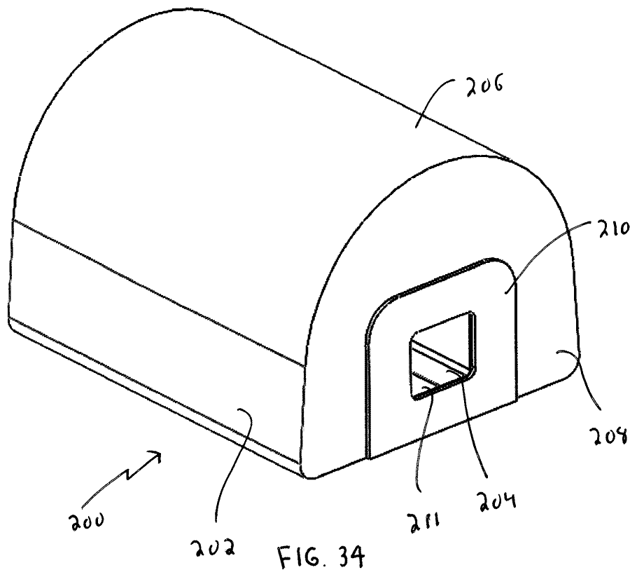

During surgery, such as arthroscopic or reconstructive knee surgery, like ACL repairs or meniscal procedure, a doghouse can be used for knee distraction to separate the knee such as shown in FIGS. 32 and 33. The distraction device includes a platform or base B to secure the foot of the patient, a frame F extends from the platform which supports a doghouse shaped support D which is underneath the knee and supports the knee. Straps can be used to secure the leg at different points to ensure alignment and immobilization of the knee during the surgical procedure. A tool E with a square shaft H fits within the opening in the doghouse D. Squeezing of lever G of tool E moves the rod R of the tool axially (up) to move the square shaft H to separate the leg and calf portions (see detail of FIG. 32). The doghouse used in the prior art device of FIGS. 32 and 33 has the disadvantage of flash that comes off the mold. This flash refers to the excess material that leaks or seeps out of the edges of the mold during the molding process, and needs to be removed, e.g., trimmed or polished. The presence of flash can interfere with the product's function, reduce its durability by creating weak points in the material, and provide unfinished edges which pose a risk of injury to the patient.

The present invention provides an improved doghouse that does not suffer the disadvantages of flash. The doghouse utilizes the design/configuration of shoulder support 90 discussed above, preferably made of a high density foam, instead of doghouse D shown in FIGS. 32 and 33. The doghouse 200 of the present invention has an arcuate upper surface 206 and an opening/channel 204 extending longitudinally therein to allow for compression of the doghouse 200, and dimensioned to receive the square shaft H. (In alternate embodiments, it can have a sleeve or other material to fill the channel).

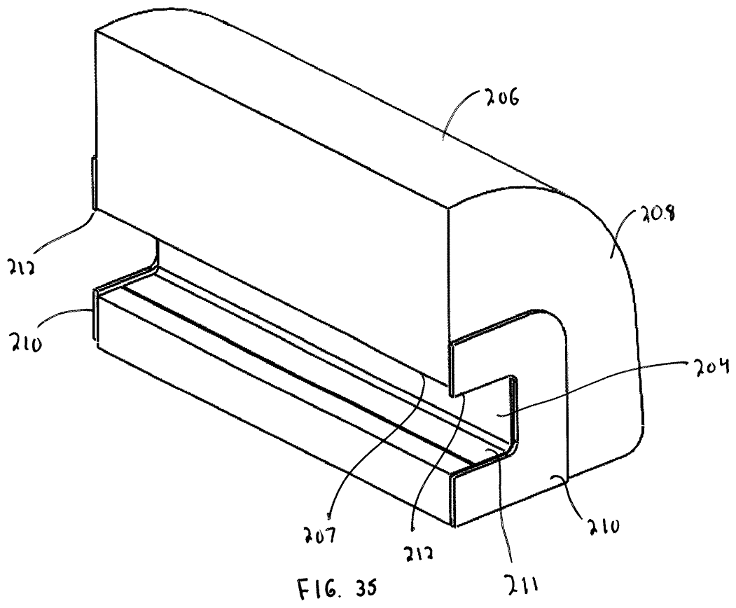

A square plate 210 (or other shaped plate) is attached to the front surface 208 of doghouse 200. It can be attached by various methods such as by glue or adhesive. The square plate 210 has an opening/center slot 211 to align with the opening 204 of doghouse 210 to enable passage therethrough of the square rod H, however, in preferred embodiments, the opening 211 is slightly offset from the opening 204 of doghouse 200 as shown in FIG. 35 to form an extension or lip 212 which extends beyond edge 207 of doghouse 200. In this manner, the lip 212 can engage a slot in the square shaft H to keep doghouse 200 in place, i.e., limiting left and right (lateral) movement. In other words, instead of being flush with the opening 204 of doghouse 210, a portion of the plate 210 extends beyond the perimeter of the opening 204 to form the lip/extension so it can fit into the slot in the square shaft H. In the illustrated embodiment, the lip 212 is only on the top of the square plate 210, however, in alternate embodiments it can in addition or in lieu of be on the bottom, left side and/or right side of plate 210. Note a second square (or other shaped) plate 210, with the offset/lip 212 and identical or similar to plate 210, can be attached to the opposite side of doghouse 210. Note doghouse 200 can be the same or of a different size than that used for the shoulder support 90. The central slot 204 can be of a different size/diameter than that shown in the Figures.

Thus, the doghouse 200 of the present invention is configured to assist distraction and is composed of a foam material and includes an arcuate upper surface 206, a longitudinally extending opening 204 and a front metal plate 210 attached to a front surface of the doghouse 200. The metal plate 210 can be offset from the opening in the doghouse 200 to form an inwardly extending lip 212 configured and dimensioned to fit in a slot of a surgical distracting tool. The distracting tool can have slot to receive the lip 212. The doghouse 200 can further include a rear metal plate 210 which can in some embodiments, be offset like the front metal plate 210 to form a second inwardly extending lip configured and dimensioned to fit in the slot of the surgical distracting tool.

The doghouse of the present invention can also be used in other surgical procedures besides those requiring knee distraction. The present invention thus includes an apparatus and method used for knee distraction or other procedures.

While the above description contains many specifics, those specifics should not be construed as limitations on the scope of the disclosure, but merely as exemplifications of preferred embodiments thereof. Those skilled in the art will envision many other possible variations that are within the scope and spirit of the disclosure.

Although the apparatus and methods of the subject invention have been described with respect to preferred embodiments, which constitute non-limiting examples, those skilled in the art will readily appreciate that changes and modifications may be made thereto without departing from the spirit and scope of the present invention as defined by the appended claims.

Additionally, persons skilled in the art will understand that the elements and features shown or described in connection with one embodiment may be combined with those of another embodiment without departing from the scope of the present invention and will appreciate further features and advantages of the presently disclosed subject matter based on the description provided.

Throughout the above description, terms such as “approximately,” “about”, “generally,” “substantially,” and the like should be understood to allow for variations in any numerical range or concept with which they are associated. It is intended that the use of terms such as “approximately”, “about”, “substantially”, and “generally” should be understood to encompass variations on the order of 25%, or to allow for manufacturing tolerances and/or deviations in design.

Where a range of values is provided, it is understood that each intervening value, between the upper and lower limit of that range is encompassed within the invention.

Although terms such as “first,” “second,” “third,” etc., may be used herein to describe various operations, elements, components, regions, and/or sections, these operations, elements, components, regions, and/or sections should not be limited by the use of these terms in that these terms are used to distinguish one operation, element, component, region, or section from another. Thus, unless expressly stated otherwise, a first operation, element, component, region, or section could be termed a second operation, element, component, region, or section without departing from the scope of the present invention.

Each and every claim is incorporated as further disclosure into the specification and represents embodiments of the present disclosure. Also, the phrases “at least one of A, B, and C” and “A and/or B and/or C” should each be interpreted to include only A, only B, only C, or any combination of A, B, and C.

What is claimed is:

1. A patient positioning device attachable to a medical table, the patient positioning device comprising:
a) a base having a top region at a first portion, a bottom region at a second portion, a right side portion and a left portion, the base configured to overlie the table and underlie the patient to provide a support for the patient; and
b) a lumbar support removably attachable to the base, the lumbar support including a first and second channel each configured to receive and secure an arm of the patient.

2. The positioning device of claim 1, wherein the lumbar support comprises a first support member and a second support member spaced from the first support member, wherein the first channel is in the first support member and the second channel is in the second support member.

3. The positioning device of claim 1, wherein the lumbar support has a first end portion, a second end portion and an intermediate portion between the first and second end portions, the first and second end portions extending a further distance from the base than the intermediate portion to form lateral raised regions.

4. The positioning device of claim 3, wherein the first channel is in the first end portion and the second channel is in the second end portion.

5. The positioning device of claim 1, wherein the base has a right side edge and left side edge, and the first channel extends laterally beyond the right side edge and the left channel extends laterally beyond the left-side edge.

6. The positioning device of claim 1, wherein the base comprises a foam material and a hook and loop fastener friendly material, wherein the lumbar support has a hook or loop material on a bottom support to removably attach to the hook or loop on the hook and loop fastener friendly material.

7. The positioning device of claim 1, wherein the hook and loop fastener friendly material is in the form of a sheet attached in manufacture to the top region of the base to provide a smooth non-irritant patient interface.

8. The positioning device of claim 1, wherein the base comprises a foam including a colorant to reduce shifting color.

9. The positioning device of claim 1, further comprising first and second straps removably attachable to the base, each strap looping through rails of the medical table and attaching to itself to secure the base to the table.

10. The positioning device of claim 1, further comprising first and second patient securing straps, each strap removably attachable to the base and extending over the patient and attaching to itself to secure the patient to the bed.

11. The positioning device of claim 9, wherein the first and second patient straps are extendable over the patient and attaching to itself to secure the patient to the medical table.

12. The positioning device of claim 1, wherein the positioning device is packaged along with multiple other positioning devices in a vacuum sealed packaging.

13. The positioning device of claim 1, wherein the lumbar support includes at least one area of Velcro for attachment to the Velcro-friendly material.

14. A patient positioning device attachable to a medical table, the patient positioning device comprising:
a) a base having an upper surface, a lower surface, a top region at a first portion, a bottom region at a second portion, a right-side region and a left side region, the base configured to overlie the table and underlie the patient, wherein the upper surface faces the patient and the lower surface faces the table, the base composed of a foam material and a hook and loop fastener compatible material on an upper surface;
b) first and second straps attached to an upper surface of the hook and loop fastener compatible material, the straps stretchable and wrap around the medical table.

15. The positioning device of claim 14, wherein the hook and loop fastener compatible material is integrated with the foam.

16. The positioning device of claim 14, wherein the hook and loop fastener compatible material is adhesively secured to the foam.

17. The positioning device of claim 14, wherein the hook and loop fastener friendly material is in the form of a sheet attached in manufacture to the upper surface of the base to provide a smooth non-irritant patient interface.

18. The positioning device of claim 14, wherein the first strap has a hook or loop fastener adhering at each end for attachment to each other.

19. The positioning device of claim 16, wherein the first and second straps are configured to be wrapped around the patient and have hook and loop fastener adhering material at each end for attachment to each other.

20. The positioning device of claim 16, wherein the foam is a non-memory foam.
